(19) United States
(12) Patent Application Publication
(10) Pub. No.: US 2010/0190265 A1
(43) Pub. Date: Jul. 29, 2010

(54) FLUIDICS DEVICE FOR ASSAY

(75) Inventors: Martin Dufva, Hornbaek (DK); Sarunas Petronis, Gothenburg (SE); Jesper Petersen, Jyllinge (DK)

Correspondence Address: EDWARDS ANGELL PALMER & DODGE LLP, P.O. BOX 55874, BOSTON, MA, 02205, US

(73) Assignee: TECHNICAL UNIVERSITY OF DENMARK, Lyngby (DK)

(21) Appl. No.: 12/095,627

(22) PCT Filed: Dec. 1, 2006
(86) PCT No.: PCT/DK2006/050073

§ 371 Date: Apr. 15, 2010

Related U.S. Application Data
(60) Provisional application No. 60/741,589, filed on Dec. 2, 2005.
(60) Provisional application No. 60/847,332, filed on Sep. 25, 2006.

(51) Int. Cl. G01N 33/53; G01N 33/00; C12M 1/00

(52) U.S. Cl. 436/501; 422/68.1; 435/287.1

(57) ABSTRACT

The present invention relates to a device for use in performing assays on standard laboratory solid supports whereon chemical entities are attached. The invention furthermore relates to the use of such a device and a kit comprising such a device. The device according to the present invention is adapted to receive one or more replaceable solid support(s) (40) onto which chemical entities (41) are attached, said device comprising a base (1, 60, 80, 300, 400, 10, 70, 140, 20, 90, 120, 150, 30, 100), one or more inlet(s) (5), one or more outlet(s) (6). The base and the solid support (40) defines, when operatively connected, one or more chambers (21) comprising the chemical entities (41), the inlet(s) (5) and outlet(s) (6) and chambers (21) being in fluid connection. The device further comprise means for providing differing chemical conditions in each chamber (21).

### FIELD OF THE INVENTION

The present invention relates to a device for use in performing assays on standard laboratory solid supports whereon chemical entities are attached. The invention furthermore relates to the use of such a device and a kit comprising such a device.

### BACKGROUND

In the detection and analysis of chemical entities based on preferential binding it is often desirable to use the optimum assay conditions, however, determination of these conditions can be laborious, especially when many parameters are involved (temperature, chemical conditions, etc.). Furthermore, the possibility to conduct different analyses of the same sample is often required, especially after the emergence of microarrays comprising multiple capture structures for the analysis of complex samples.

This is relevant for example in the field of microarrays. Microarrays are well known in the art and are frequently used for example for gene expression monitoring and single nucleotide polymorphism (SNP) analysis. Microarrays can however also include tissue samples, proteins, or other complex chemical entities. Microarrays exploit the preferential binding of complementary chemical entities, for example single-stranded nucleic acid sequences. The chemical entities attached to the microarray are usually called capture probes, and the complementary chemical entity is called an analyte or a sample material. “Chemical entities” also include organic molecules such as biomolecules (DNA, RNA protein, lipids), small molecules (drugs and pesticides) and mixtures of different biomolecules or small molecules. A microarray typically comprises a glass (or some other material) slide, on to which the known chemical entities are attached at predetermined locations (spots). There may be tens of thousands of spots on an array, each containing a large number of identical chemical entities, or fragments of identical chemical entities. The spots are printed on the microarrays by a robot, synthesized by photo-lithography or deposited by ink-jet printing.

Microarrays are typically assayed by applying a sample solution containing analyte molecules on the microarray. The specific binding of the analyte to the capture probes on the array take place. Analyte not specifically adsorbed or attached is removed by stringent washing. The type and amount of analyte present in the sample is then determined by detecting the location and amount of specific binding on the microarray spots. The detection is performed by pre-labeling the analyte or post-labeling the analyte-capture probe complexes (fluorescent, colorimetric, magnetic or radioactive labeling) or by label-free analysis (SPR, QCM, AFM etc). Usually analysis of chemical entities using microarray technology requires a number of different analytical instruments and technologies, like fluorescence scanners and image analysis software.

Microtiter plates are another example of a standard product used for multiplex analysis of chemical entities whereby chemical entities can be coupled to the surface of the microtiter plate at specific locations (wells). Analysis of microtiter plates employs a number of different instruments for the various steps of the procedure, e.g. a washing station, hybridisation station, a scanner etc.

The bottle neck of microarray- and microtiter plate-type technologies is however that they require extensive optimization, i.e. their specificity is influenced by the environmental conditions under which binding of the analyte molecules to capture probes takes place. The optimal environmental conditions, such as temperature and chemical composition of the reaction buffer, are individual for each target probe pair and might vary significantly dependent on the size and structure of the molecules. The optimal binding conditions can not always be theoretically predicted (Pozhitkov, A., et al., Tests of rRNA hybridization to microarrays suggest that hybridization characteristics of oligonucleotide probes for species discrimination cannot be predicted, Nucleic Acids Res, 2006, 34(9): p. e66.), and hence their experimental determination is time consuming. Furthermore, when designing the microarray or microtiter plate based assays, only those capture probes which require similar environmental conditions for specific binding can be included in the single microarray slide or microtiter plate, otherwise the specificity and sensitivity of the assay is lost.

Allele specific hybridization to DNA microarrays has been shown to be a powerful method for high throughput genotyping (Matsuzaki, H. et al. Genotyping over 100,000 SNPs on a pair of oligonucleotide arrays. Nat Methods 1, 109-11 (2004).). A difficulty associated with allele specific hybridization for microarray-based genotyping is however, that the method depends on the type of mutation (substitution and insertion/deletion) and the sequences surrounding the mutation (Syvanen, A. C. Toward genome-wide SNP genotyping. Nat Genet 37 Suppl, S5-10 (2005). When a probe-set is design for a DNA microarray, probes are often to be used at only one condition, e.g. the same post-hybridization wash. Therefore, all probes are designed in silico to have the same melting temperature Tm. Due to difference in base-pairing between the base pairs A-T and G-C, a mutation located in an AT rich region requires longer probes sequences than one in a GC rich region, to achieve the same theoretical Tm. Another consideration when designing probes is, that long probe sequences usually have reduced ability to discriminate between mismatched and perfectly matched probe/target duplexes compared to short probes. Selection of probes is further hampered by limitations in computer assisted probe design using thermodynamic models, which are unable to precisely predict the binding strength of probe/target duplexes and effect of an introduced mismatch (Pozhitkov, A. et al. Tests of rRNA hybridization to microarrays suggest that hybridization characteristics of oligonucleotide probes for species discrimination cannot be predicted. Nucleic Acids Res 34, e66 (2006)). A possible explanation for the low prediction accuracy is that thermodynamic hybridization models are based on DNA hybridization in solution and equal amount of probe and target. This is not the case for DNA microarrays, having one end of the probe immobilized to a surface, which induces large changes in probe behavior (Vainrub, A. & Pettitt, B. M. Coulomb blockage of hybridization in two-dimensional DNA arrays. Phys Rev E Stat Nonlin Soft Matter Phys 66, 041905 (2002). Vainrub, A. & Pettitt, B. M. Surface electrostatic effects in oligonucleotide microarrays: control and optimization of binding thermodynamics. Biopolymers 68, 265-70 (2003)) Development of an allele specific hybridization assay is therefore largely a trial and error process, where many cycles of probes choice and testing are required to obtain an array of probes that all perform adequately using one washing condition after hybridization. It would therefore be desirable to create precisely defined conditions over DNA array during hybridization or during post hybridization washes. Such precisely defined conditions will (i) allow for the use of probes with different optimum conditions to be located in the same array and (ii) decrease the time to find optimum assay condition for a set of probes. Post hybridization wash of separate slides at different condition is the easiest to implement, however it requires one slide per washing condition which can be cumbersome in a development process as well as in the diagnostic situation. Optimization of a DNA array for detection of 100 different mutations requires a minimum of 800 slides assuming eight different conditions to be tested and a heterozygote DNA sample for each mutation.

Alternatively, temperature gradients can be created on a single slide by varying the temperature over time, also called temporal temperature gradients (Noerholm, M., Bruus, H., Jakobsen, M. H., Telleman, P. & Ramsing, N. B. Polymer microfluidic chip for online monitoring of microarray hybridizations. Lab Chip 4, 28-37 (2004). Anthony, R. M. et al. Effect of secondary structure on single nucleotide polymorphism detection with a porous microarray matrix; implications for probe selection. Biotechniques 34, 1082-6, 1088-9 (2003)) or dynamic allele-specific hybridization (DASH) (Howell, W. M., Jobs, M., Gyllensten, U. & Brookes, A. J. Dynamic allele-specific hybridization. A new method for scoring single nucleotide polymorphisms. Nat Biotechnol 17, 87-8 (1999). Jobs, M., Howell, W. M., Stromqvist, L., Mayr, T. & Brookes, A. J. DASH-2: flexible, low-cost, and high-throughput SNP genotyping by dynamic allele-specific hybridization on membrane arrays. Genome Res 13, 916-24 (2003)). As opposed to DNA oligonucleotide microarrays, DASH has the target immobilized on the microarray substrate and subsequently investigates duplex formation using labeled probes, which are first hybridized and subsequently de-hybridized by a temporal temperature gradient. Precise melting curves are thereby obtained, providing precise and robust genotyping. However, only few probes can be investigated in DASH compared to DNA microarrays. Other drawbacks associated with temporal temperature gradients are that fluorochromes, used to monitor hybridization events, change quantum efficiency with temperature (Noerholm, M., Bruus, H., Jakobsen, M. H., Telleman, P. & Ramsing, N. B. Polymer microfluidic chip for online monitoring of microarray hybridizations. Lab Chip 4, 28-37 (2004)) as well as fluorochromes might bleach during the assay. Such changes of fluorescence as induced by heat obscures the results and must be accounted for. Furthermore, temporal temperature gradients require specialized complex equipment that usually has a relative low throughput, and only allows quantification of rather small arrays. The reason is that a solid support is mounted into the device and, depending on optical system, a rather small array can be investigated if not a scanning device is utilized. A picture is taken of the device at particular temperature (e.g every 0.5° C. or every 1° C.). Such devices typically have a temperature controller, fluorescent detection device, in combination with some optics. They may also have scanning capacity. Because of the need for a relatively expensive detection unit, the device usually handles only one sample at a time.

Another mean of creating temperature gradients is by creating a spatial gradient over a surface thereby avoiding the negative effects of gradients such as limited space for arrays, low throughput and changing quantum efficiencies during assay. Kajiyama et al demonstrated a microfabricated chip with heaters that created a spatial temperature gradient over a microchip (EP 1108472 B1, US 2002/164778 , U.S. Pat. No. 6,428,749, U.S. Pat. No. 6,346,383. Kajiyama, T. et al. Genotyping on a thermal gradient DNA chip. Genome Res 13, 467-75 (2003)). DNA microarrays where immobilized on pads and each pad was provided with individual thermal control. A gradient of different thermal conditions could thereby be created over the chip. However these chips are difficult to fabricate, they are difficult to provide with chemical entities (spots) and difficult to scan after hybridization and washing. Furthermore, the complex chip can only be used for a single hybridization experiment, which is not cost efficient.

Micro arrays in closed micro fluidics system can be treated with infrared light so that a spatial temperature gradient is created over a chip or solid support (US 2005/0164407 A1). The described device is build over the Geniom One from FEBIT that utilizes an array of mirrors to synthesize DNA on chip. The same mirrors are subsequently used to guide infrared light thus heating the chip. The mirrors can be controlled so that different part of the chip is heated differently. The hybridization or dehybridization can be monitored temporally or spatially using the device. However, this device is based on a specific micro array support format that is not normally or most commonly used by laboratories around the world. Furthermore, the machine can only process about one chip per day making it too slow for most applications. Mao et al demonstrated a device for creating linear temperature gradients over a micro array produced on microscope slides (WO 2003037514 [A3], Mao, H., Holden, M. A., You, M. & Cremer, P. S. Reusable platforms for high-throughput on-chip temperature gradient assays. Anal Chem 74, 5071-5 (2002)). The device forms a hot and a cold side with the micro array in between creating a linear temperature gradient over the array. A downside of this solution is however that micro arrays have to be fitted into the small channels, this can be challenging and only small arrays can be treated with different conditions given the limiting channel size. Another drawback is that the system is not flexible in terms of choice of gradients. For instance non linear gradients are difficult to make because the location and the micro fluidics must be adjusted to the temperatures in the gradient. It would be much better to be able to control the temperature in each channel using software.

The solution could also be used to create a chemical gradient over the array by letting two buffers with different composition meet. After the meeting point the buffers is mixed by diffusion and thereby creating a chemical gradient. The gradient is sampled using different thin channels. Again a real drawback of such solution for microarray analysis is the lack of flexibility to control the stepwise gradient. It is possible to combine a chemical and thermal gradient by applying the chemical gradient in one dimension and the thermal in another dimension. It is questionable if such a micro fluidics solution is robust enough to be used in diagnostics of professionally for microarray developments.

It is the object of the invention to provide a simple and reusable device for performance of multiconditional assays on a macro-, micro- or nanoscale. That e.g. a channel is a micro channel is defined by the fact that at least one dimension of the channel is <1 mm which results in a high Reynolds number.

It is a further object of the invention to provide a device which can facilitate the analysis and optimization of at least one parameter defining the assay conditions of (a) given molecule(s) or chemical entities in a given assay.

It is a further object of the invention to provide a device which can treat large arrays of chemical entities under different and exactly defined conditions.

It is yet another object of the invention to provide a device which can perform many different assays with improved sensitivity. Such a device can also be used to improve dynamic range of the colorimetric readout of an assay.

It is a further object of the invention to provide a device which can process many different samples at the same time

It is a further object of the invention to provide a device which can function together with the most widely used micro array platforms such as microscope slides and proprietary formats such as the format used by Illumine.

### SUMMARY OF THE INVENTION

The present invention relates to a device for performing an assay. The device is adapted to receive one or more replaceable solid support onto which chemical entities are attached. The device comprises a base, clamping means for pressing the base against the solid support, one or more inlet(s), one or more outlet(s); the base and the solid support are when operatively connected defining one or more chambers, the inlet(s), the outlet(s) and the chambers are being in fluid connection; and the device has means for providing a spatial gradient of a parameter within one chamber or between two or more chambers.

This device is a significant improvement to other comparable devices for the following reasons:—it allows for a large number of probes to be analyzed,—it is simple to operate,—it is reusable,—it is easily made compatible with scanners, spotters and other micro array specific hardware already present at the market,—it does not depend on a specific array substrate or solid support,—it allows for both linear and non-linear gradient of both chemical conditions and temperature or a combination of these parameters,—it can be used to expand the dynamic range of colorimetric detection.

It has been discovered that by providing a reusable device including a fluidic network and/or a micro-heater network, it is possible to combine the device with any solid support of compatible dimensions for operative assembly in the device and to perform an assay on the chemical entities attached to the solid support or the chemical entities in a fluid in a simple fashion.

As the device is reusable, after performing the assay, the solid support can be removed and the device washed and reused for the same or a different assay.

The invention also relates to the use of such a device in an assay, and a kit comprising the device together with a solid support onto which chemical entities are attached.

### DETAILED DESCRIPTION

Developing assays for detection and analysis of chemical entities can be a difficult and laborious task, because there may be a large number of variable parameters that need to be optimized. Although some of these parameters, for example the melting temperature of a DNA-strand, can be predicted in silico on the basis of algorithms, the predicted values are not always reliable (Pozhitkov, A., et al., Tests of rRNA hybridization to microarrays suggest that hybridization characteristics of oligonucleotide probes for species discrimination cannot be predicted. Nucleic Acids Res, 2006. 34(9): p. e66.).

A device according to the present invention provides an easy and flexible platform for conducting an assay of chemical entities and determining optimum assay parameters. The device is capable of receiving one or more solid supports and is easily adapted to receive different types of standard solid supports such as glass slides, microarrays, and microtiter plates.

After the immobilisation of one or more chemical entities on a solid support, the solid support can be inserted into the device and assayed. As the device is capable of providing isolated assay conditions on the surface of the solid support, it is possible to test various parameters or combination of parameters on the one and same slide and in a single experiment. Accordingly, the labour time for determining the optimum conditions for conducting an assay is considerably reduced.

The device comprises a base, which provides one or more chambers when operatively connected to the solid support. In the embodiments of the figures the base forming at least part of the chambers are created by joining together several layers of different material and shape. Analytical assays may be performed on chemical entities attached to the surface of the solid support which is exposed to the one or more chambers. By varying the assay conditions (parameters) within the one or more chambers the chemical entities on the solid support can be exposed to a range of assay conditions.

According to one embodiment of the invention the parameter that can be varied in the fluidic device is the chemical composition of the fluid passing through the channel e.g. the chemical composition of the washing buffer.

If the device comprises a fluidic network for providing a fluid concentration gradient, the fluid can be liquid or gas and the solution can for example be based on organic and inorganic molecules, small chemical compounds, pharmacological agents, biochemical compounds, cells or cell parts, etc.

According to one embodiment, the device is connected to one or more vessels each having a liquid with an exactly defined chemical composition. This use of predefined compositions is paramount for good diagnostics. In such embodiments, the gradient can be created by directing fluids with predefined concentration gradients from a series of reservoirs to each of the chambers in the device i.e. the concentration gradients are not made after entering the device.

The range of the temperature gradient used is dependent on the type of assay to be performed in the device. For example nucleic acid hybridisation or de-hybridization assays may be optimized with respect to the temperature and the length of the nucleic acid probe. Temperature gradients may also be used to examine the effect of temperature on protein folding and/or function.

The device can comprise one or more temperature controllers for providing an, exactly defined temperature at the surface of one or more of the solid supports when incorporated in the device. The temperature variation can be a spatial temperature gradient over the surface of the solid support, i.e. a spatial temperature gradient over a number of chambers, or a spatial temperature gradient within one chamber. When the spatial temperature gradient is formed in a number of chambers, each chamber is capable of accommodating a fluid having one temperature. The gradient is apparent when the chambers are seen in relation to each other. This gradient can be kept constant for as long time as required by the performed assay.

A spatial temperature gradient is a change in temperature over a space, for example within a chamber or from chamber to chamber.

The temperature gradient can be obtained by a differential heating/cooling of each chamber. For example heating can be obtained by electric heating, radiation heating, and heating by a fluid etc. Cooling can be provided for example with a Peltier element or by heat exchange with circulating pre-cooled fluid.

One of the advantages of the invention is that the temperature regulator(s) is/are reusable and can be controlled, i.e. the temperature gradients can be varied independently of the assay to be performed.

According to one embodiment the temperature-regulator is made of an electrically conductive material of a certain resistance, e.g. metal or alloy, conductive ceramic (e.g. indium-tin-oxide) or a conductive polymer positioned in connection with each chamber. The temperature-regulator can also be made of a thermoelectric element, such as a Peltier element. The Peltier elements can both heat up or cool down the chambers so they can be used in the device for creating thermal gradients below the ambient temperature.

According to yet another embodiment, the temperature-regulator is a heater of a resistive metal or alloy components. The metal or alloy components can be of any metal or alloy, e.g. iron, copper, platinum, aluminium, nickel, gold, wolfram, tungsten, and chromium metals or alloys. Copper is useful in cases where the part of the device containing the heaters is a part of a printed circuit board (PCB) used in the electronic industry. This way each heater can be directly integrated with e.g. controlling electronics or other electrical components of the device. The metal components can be in the form of wires, etc.

According to one embodiment, the resistive metal components can be in the form of a wire, coil, plated planar structure or a thin film. When resistive metal or alloy components are used they can be located in the base and provide heat to the area of the base corresponding to the areas forming the chambers. The resistive metal components can be formed in such a way that they provide local heating in the chambers and no heating outside the chambers. This can be achieved for example by adjusting the geometrical shape of the resistive wires or thin films. A base with a wire having a small diameter in the areas defined by the chamber would have higher resistance and therefore provide Joule heat therein. The wider diameter wire outside the areas defined by the chambers would have low resistance and serve as contact leads between the heaters and controlling electronics or power source.

According to yet another embodiment, localized temperature regulation can be achieved by heat exchange with remotely pre-heated or pre-cooled fluids (e.g. air or water) directed to specific parts of the chip by e.g. channels.

Each chamber can be provided with an individual temperature regulator, i.e. the temperature provided by the temperature regulator in each chamber can be controlled separately. Hence, the user of the device can determine the temperature in each chamber independent of the surrounding chambers within the limits determined by the conductive heat transfer in the device materials and solid support. if the temperature is too low in one chamber it can be heated without influencing significantly the temperature of any other chamber. This construction of the device provides a possibility of having spatial temperature gradients over the surface of the solid support, in each chamber or from chamber to chamber.

When the temperature regulators are located in the base, as described above, the spatial temperature gradient over the surface of the solid support or from chamber to chamber can easily be obtained.

In addition to a temperature regulator, the device can also include one or more temperature sensor(s). The temperature sensors can measure the temperatures of fluid that may be introduced into one or more chambers in the device. The temperature regulators may have a corresponding temperature sensor. The information from the sensor can then be provided to a control mechanism, for example computing means, which by way of a program uses the information to regulate the temperature regulators. The control mechanism can also be a dedicated electronic circuit or a thermo-mechanical mechanism well known in the art. This combination of temperature regulator and temperature sensor allows the temperature in the device to be controlled, monitored and logged.

The electronic control is typically based on a feedback loop, i.e. the device measures the actual temperature, compares it with the required temperature (set-point) and adjusts the electric power to the heater according to an algorithm. This algorithm can be for example proportional (P), proportional integrative (PI), proportional integrative differential (PID), fuzzy-logics.

The temperature sensor can be made of the same material as the temperature regulator, but it can also be made of a different material. The temperature sensor can be a resistive wire, thermocouple, thermistor or other similar devices. The temperature sensor might be part of the temperature regulator itself, or it might be a separate component. The temperature sensor is usually not included in the temperature regulator, but is a separate component. This allows measuring of the temperature in the functional part of the heated/cooled object.

The fluidic device, providing a temperature gradient array, can be used for various purposes, for example optimizing and performing precise genotyping of SNPs and other small mutations, where the device provides the optimal reaction conditions for each genotyping probe to be determined and used, making probe design simple and inexpensive.

This device can also be used to determine the melting points of immobilised capture probes-analyte hybrids in high throughput fashion.

Chemical gradients can be made at a specific steady temperatures maintained by temperature regulating mechanism. This eliminates variation of overall assay conditions within a microarray due to changes in ambient temperature because of e.g. day-night cycle or season change.

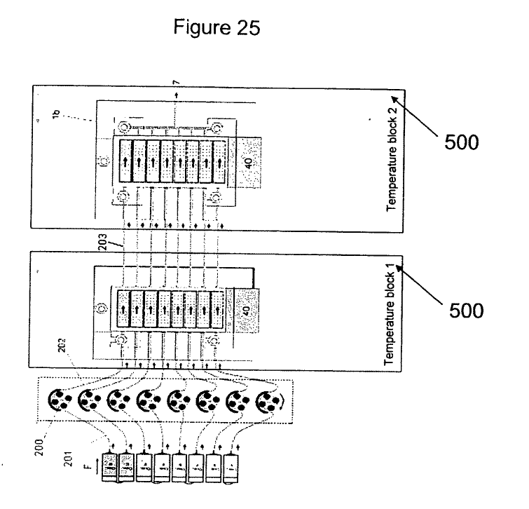

The steady temperature can be achieved by enclosing the entire device including the microarray on support, base, gradient creating microfluidic system, pumps, reservoirs of chemicals in an environmental chamber i.e. chamber made of thermally insulating materials and having active temperature control of the air within. Alternatively, the steady temperature can be achieved and controlled only on the support and the base of the device by using integrated temperature controllers in the base. Temperature regulation would be achieved by integrated temperature controllers such as resistive elements, thermoelectric elements, heat exchange with thermally pre-treated fluids, integrated temperature probes such as thermistors, thermocouples, resistive elements, and temperature regulation feedback loops e.g. P, PI, PID, fuzzy-logics algorithms. When using more than one base provided with differing chemical conditions in each chamber and integrated temperature regulation, it is possible to have steady and different temperatures for each base and respective solid support provided with an array (FIG. 25). Such set-up allows combination of multiple chemical and thermal conditions on a single device, which is beneficial when screening the optimal microarray assay conditions. (see also below description of combined chemical and thermal gradients)

The fluidic device, providing a chemical gradient array, can be used for various purposes, for example optimizing and performing precise genotyping of SNPs and other small mutations, where the device allows the optimal reaction conditions for each genotyping probe to be determined and used, making probe design simple and inexpensive. The fluidics device allows for use of probes with widely different operation optimum in terms of chemical composition of e.g. buffer and temperature in the same array. The probes can be arranged in replicates, each replicate receiving different conditions. This configuration is very useful for optimization and diagnostic assays. The probes can also be organized according the previously determined optimal working condition. Such arrays are particularly useful for large assays where more than 5000 mutation is investigated.

The combination of multiconditional stringent washing, i.e. stringent washing where e.g. the chemical composition of the washing buffer or the temperature is varied on different places of solid support, with colorimetric method gives an advantage over a standard colorimetric method performed in one stringent washing condition.

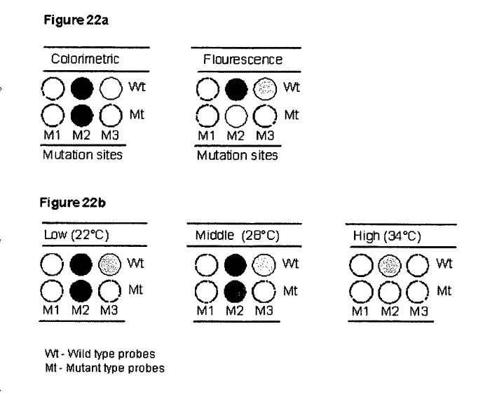

Multiconditional stringency wash eliminates detection problems arising from low dynamic range of the colorimetric method. If for example at one condition two spots of the array are so bright after having been subjected to colouring (“overexposed”) that they cannot be quantitatively compared to each other as they are e.g. just white, the spots at another position on the solid support where washing conditions were inducing higher stringency (e.g. 0.3×SSC+0.5% SDS at 22° C. corresponds to low stringency and 0.3×SSC+0.5% SDS at 34° C. corresponds to high stringency), will not be overexposed but will show grey-tones which can be used for quantitatively comparing the spots (FIG. 22). Similarly, spots which were week and maybe not visible at all at one part of the solid support where stringency of the wash was high, could become visible at another part of the solid support having been exposed to conditions of lower stringency during the washing. Thus, on one and the same solid support it is possible to quantitatively compare e.g. pairs of mutant versus wild type genes representing both very strong and very week spots, just in different spatial positions of the array corresponding to different washing conditions. This method can e.g. be used for very sensitive mutation detection or SNP detection without the need of a fluorescence scanner which scanner although having a wide dynamic detection range and being able to quantify the entities properly during a single washing condition, also is very expensive.

The device according to the invention can also be used for automated generation of a standard curve for quantitative competitive microarray-based immunoassays.

Basically any assay employing a solid support using a chemical gradient can be performed in a device according to the present invention.

It is for example possible to determine the IC50 dose for various interacting chemical entities such as a drug-target interaction. The IC50 represents the efficacy of a drug or inhibitor, where IC50 is the concentration required to produce 50% inhibition. In this case, a dilution series of the drug is created with which the target molecules attached to the solid support are in functional contact. Thereby the array within each of the one or more chambers of the device can be monitored to detect specific inhibition of one or more target molecules attached to the array. When many similar potential target chemical entities are to be screened, these can be arrayed on the solid support for simultaneous assay with the possibility to detect the inhibitory effect of a drug and its specificity.

It is also possible to determine binding constants for different protein-protein, protein-drug, protein-carbohydrate, protein-lipid interactions, using a device according to the invention.

According to a further embodiment of the invention the device is capable of simultaneously generating series of conditions for two different parameters, to form a matrix of conditions to which the surface of the solid support is exposed.

In this way the device can be used to monitor the combined effect of different reaction conditions in a matrix format. This matrix format can be used for example to determine the optimum combination of parameters for a specific assay. An example is to use temperature as one parameter and a chemical condition as the other. This can be used for example in SNP analysis to find out the melting temperature of the oligonucleotides as well as the optimum salt concentration. Another possibility is to provide two different chemical gradients in order to find the optimum chemical conditions for an assay.

According to one embodiment of the invention the assay performed in the device is a bioassay. Many different bioassays are possible, e.g. nucleic acid (DNA/RNA) hybridisation, protein interactions with various chemical entities e.g. antibody-antigen interactions, protein-protein, protein-drug, protein-carbohydrate, protein- lipid interactions, the interaction between a drug and another molecule, etc. Furthermore, tissue arrays, cell arrays and other types of arrays containing polymers are also eligible for use in the device. These assays also relate to the interactions of larger units, e.g. viruses, cell parts, etc.

The bioassay can be based on one or a combination of biochemical compounds selected from the group consisting of proteins, peptides, oligonucleotides, carbohydrates, polysaccharides, lipids, antibodies, antigens, cells or cell parts.

Bioassays are especially useful in SNP-analysis, genome expression monitoring, DNA-fingerprinting, in hormone analysis (e.g. growth hormones), allergy studies, detection of steroids, risk profile testing, etc.

According to another embodiment of the invention the assay performed in the device is a chemical assay. Such chemical assays include analysis of organic or inorganic chemical compounds e.g. in gas or liquid phase. These assays are especially useful in the analysis of gas samples and the detection of for example detergents, pesticides, herbicides, fungicides and various factors of pollution.

The chemical assay can be based on one or more compounds comprising pesticides, pollution, toxic materials, explosive materials, etc.

According to one embodiment of the invention the device can perform an assay in the following manner. The device first is used to create multiple environmental conditions for capturing analyte to the chemical entities on the microarray. After the capturing step is finished, stringent washing is performed over the solid support to remove unspecifically captured analyte, followed by analysis of the locations and amount of the remaining bound analyte on the solid support.

According to another embodiment of the invention the device can perform an assay in the following manner: First the capture is performed in the device under one condition and then stringent washing is performed under multiple environmental conditions in the device.

This embodiment is preferable for cases where the solid support is for example a DNA-microarray in order to minimise the volume of the assay fluid. During the stringent washing step, the microarray is constantly flushed with a washing buffer, which is available in large volumes and is relatively inexpensive. The device distributes washing buffer to the chambers so that concentration and/or temperature of the buffer is constant within individual chambers and varies from chamber to chamber, preferably as a linear gradient. This causes the analyte to remain bound i.e. captured or to dissociate from the chemical entities and to be washed away depending on the strength of the binding and on the conditions in the particular chamber.

Thus the device can be used for example to:
1) determine dissociation/melting curves and melting point temperatures of multiple analyte-capture DNA hybrids in a single experiment on a single microarray slide,
2) determine under which conditions multiple analytes remain bound to their complimentary chemical entities but are dissociated from all non-complimentary chemical entities, which defines optimal assay conditions in a single experiment and on a single microarray slide,
3) determine under which conditions multiple wild type analyte DNA can be discriminated from mutant analyte DNA strands binding to the same chemical entity in a single experiment on a single microarray slide. These optimal discrimination conditions dissociate and remove mutant DNA but preserve bound wild type DNA. The mutation can be as small as single base (SNP detection),
4) perform multiplex, highly sensitive and specific assays by arranging capture sites on a microarray so that they match the chambers providing optimal conditions (see also 2) or 3)).

However, a sample can be arrayed on a solid support and probed with a single analyte at different concentrations to characterise binding constants and cross reactivity. For instance, a microarray of receptors can be probed with different concentrations of ligands to investigate cross reactivity and binding constants in one single experiment. Similarly DNA sequences and protein arrays can be probed with different concentrations of a protein, drug or any other compound to directly identify interacting partners, binding constants and cross reactivity with the chemical entity investigated.

The solid support which is inserted into the device can be selected from the group of materials comprising glass, polymers, silicon, metals, ceramics and can take the form of a microscope slide, a chip or a microtiter plate

According to an embodiment of the invention the fluidic device further comprises a sealer for providing sealing between the base and the solid support. The sealer can be made of a flexible elastic material, which deforms to a certain extent from its original shape when pressure is applied to it and therefore close gaps and provide a hermetic seal. Examples of suitable materials are e.g. synthetic or natural rubber, copolymer of butadiene and acrylonitrile, copolymer of ethylene, methyl acrylate, copolymer of vinylidene fluoride and prefluoro-propylene, silicone, such as poly-dimethyl-siloxane (PDMS silicone). The sealing means can also be provided by an adhesive, for example a tape having an adhesive surface on both sides. The sealer can be disposed after each use of the device.

According to a further embodiment of the invention the fluidic device further comprises a top part, for applying even pressure on the solid support when inserted in the device. This top part, or lid, is included in order to secure optimum application of pressure when clamping the solid support and device together and form a good seal between the solid support and the base.

According to an embodiment of the invention the base is at least partly made of a material, which is a good thermal insulator, resistant to solvents, heat resistant, possible to form to include channels and impermeable to fluid. The material can be a polymer material and the surface of the polymer material can be treated in order to prevent reaction between the fluid and the polymer material or in order to increase the flow properties inside the chambers. Suitable materials are polymethyl methacrylate (PMMA), cyclic-olefin copolymer (Topas®), polystyrene, polycarbonate, polyvinyl chloride, acrylics, polytetrafluoro ethylene, epoxy, polypropylene, polysulfone, polyethylene, nylon based polymers and co-polymers or composites. The material can also be a ceramic material, e.g. glass, silicon, porcelain or quartz. The material or the material surface can be hydrophilic or hydrophobic in order to regulate the flow pattern of the fluid in the chambers. The base can also be made of a combination of one or more of the materials listed above.

Also the chambers can be provided with one or more structured inner surfaces in order to distribute the flow, enhance mixing by creating a turbulent flow profile when fluid is flowing in the chamber. The structures can be elevated or recessed in comparison with the normal smooth inner surface of a chamber. The structures are e.g. positioned in the bottom of the chamber and can e.g. have the form of one or more rhombs, as one or more successive arrow heads (herringbone), triangles, rectangles or as bars distributed in symmetric or asymmetric manner, oriented perpendicular or skew to the flow direction.

In some cases it is preferred that the device is made of a transparent polymer, for example to allow the filling of the channels and the chambers to be monitored. However, in other cases it might be preferred that the device is made of a light absorbing/reflecting material, for example when working with light-sensitive materials such as chlorophylls or fluorescent dyes.

According to one embodiment of the invention the device is formed in a way and from a material which makes it possible to detect a signal, and the device furthermore includes means for detecting a signal.

According to one embodiment of the invention the solid support which can be inserted into the device is selected from the group consisting of glass and polymeric slides and microtiter plates.

According to another embodiment the fluidic device is reusable.

According to yet another embodiment of the invention the same device is capable of performing different fluid phase assays.

According to a further embodiment the invention comprises a kit comprising the fluidic device and a solid support onto which chemical entities are attached. The kit can also include tubings for connecting the devices to the pumps; pumps for filling and perfusing the channels and the chambers with fluid; electronics for measuring and logging temperature by temperature sensors; electronics for controlling the electric power on the temperature-regulators; electrical power source for supplying temperature-regulators, temperature sensors, and electronics; software for controlling the electronics.

The invention will now be described in more detail with reference to the drawings:

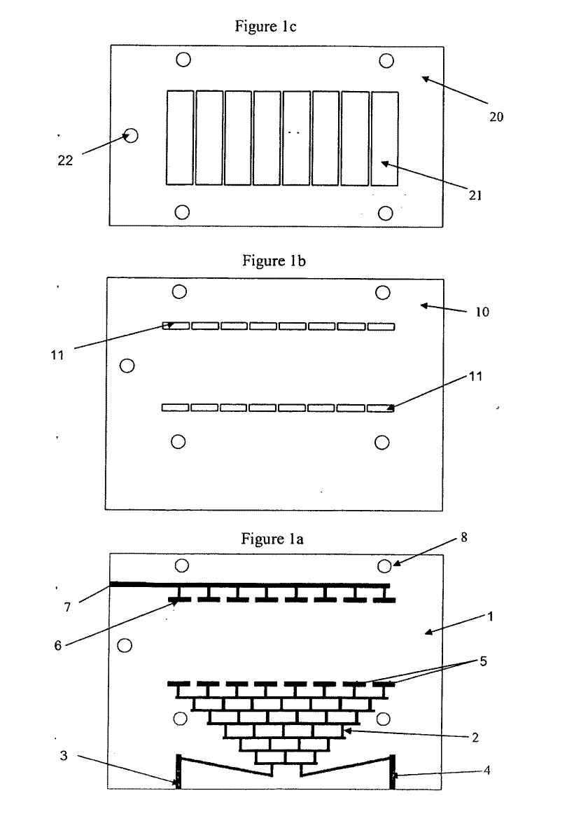

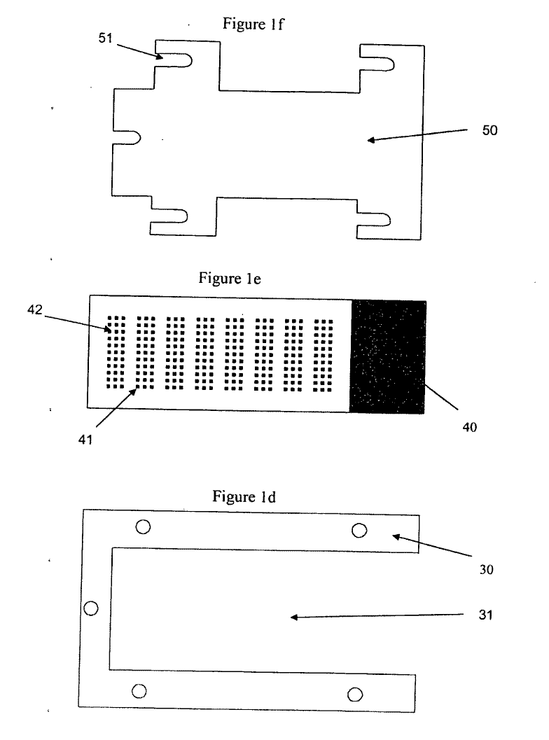

FIGS. 1a-1f show a top view of various layers of one embodiment of a device according to the invention which is capable of producing a chemical gradient.

FIG. 2 shows a top view of one embodiment of the assembled device according to the present invention, which essentially comprises the parts shown in FIGS. 1a-1f, combined with a solid support.

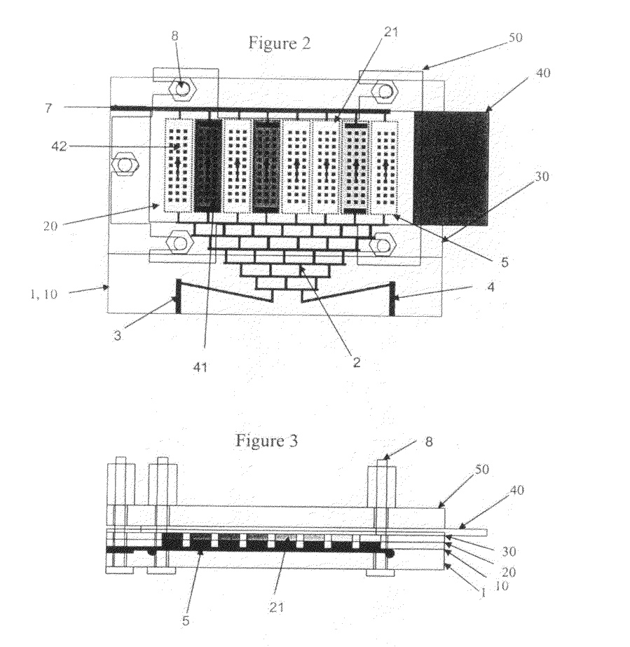

FIG. 3 shows a side view of the embodiment shown in FIG. 2.

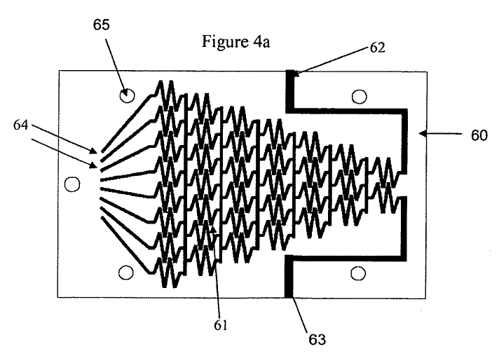

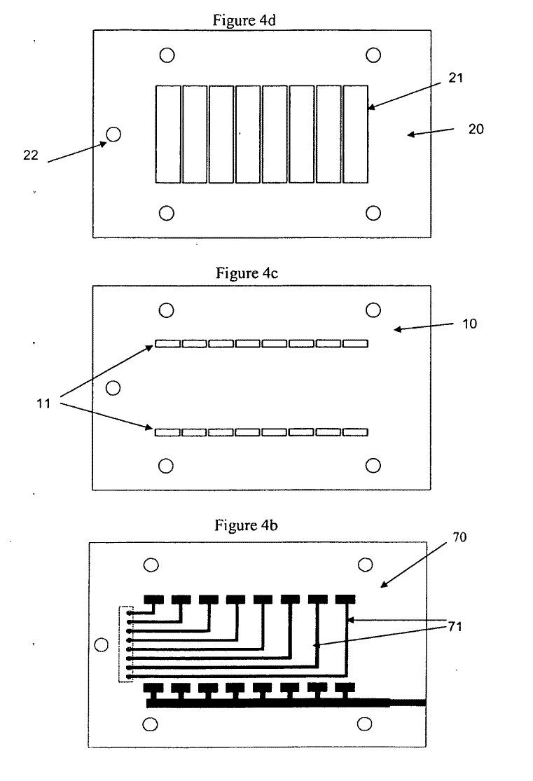

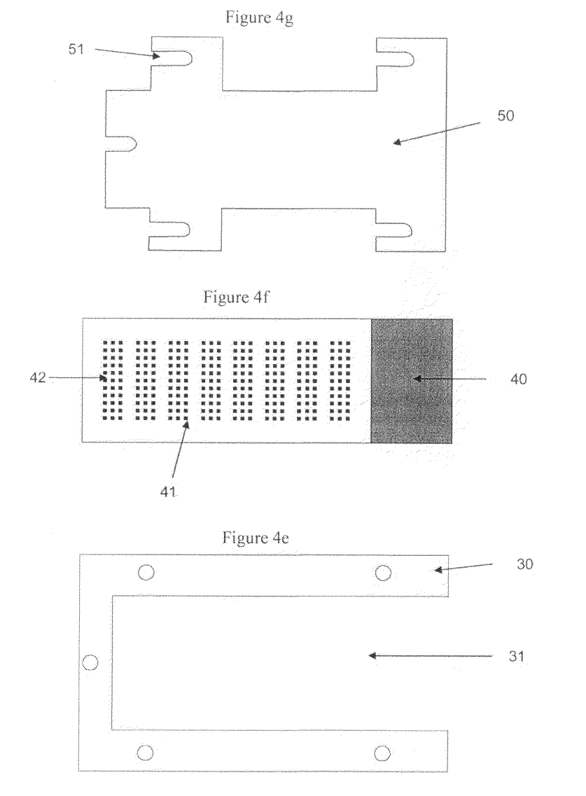

FIGS. 4a-4f show a top view of various layers of another embodiment of a device according to the invention which is capable of producing a chemical gradient.

FIG. 5 shows a top view of one embodiment of the assembled device according to the present invention, which essentially comprises the parts shown in FIGS. 4a-4f, combined with a solid support.

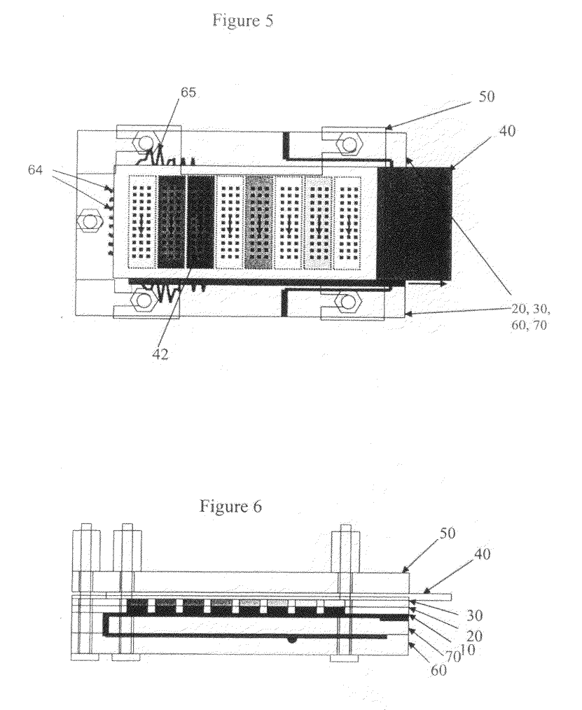

FIG. 6 shows a side view of the embodiment shown in FIG. 5.

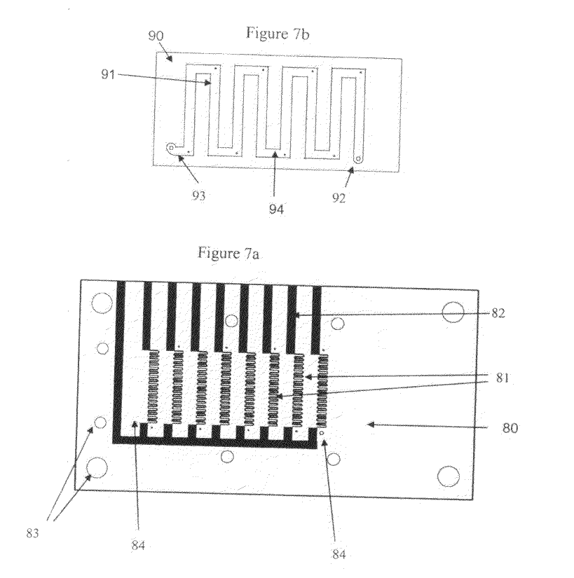

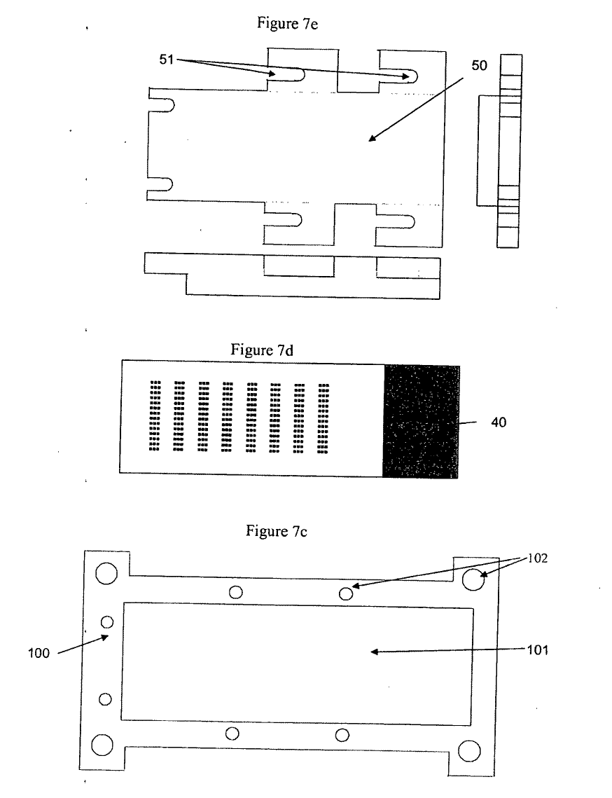

FIGS. 7a-7e show a top view of various layers of one embodiment of a device according to the invention which is capable of producing a thermal gradient.

FIG. 8 shows a top view of one embodiment of the assembled device according to the present invention, which essentially comprises the parts shown in FIGS. 7a-7e, combined with a solid support.

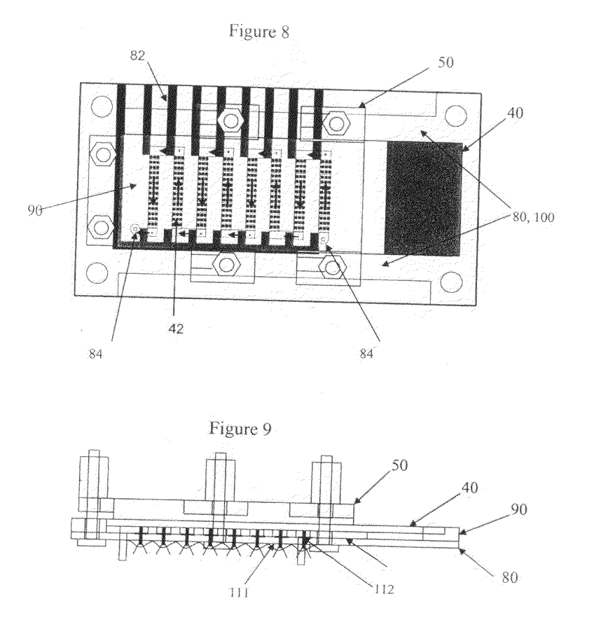

FIG. 9 shows a side view of the embodiment shown in FIG. 8.

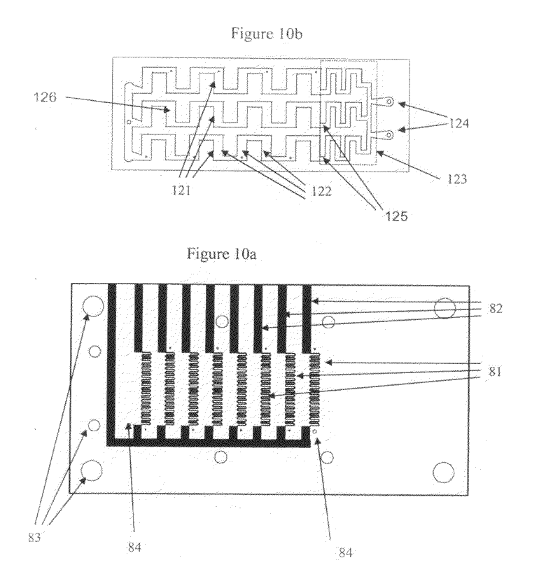

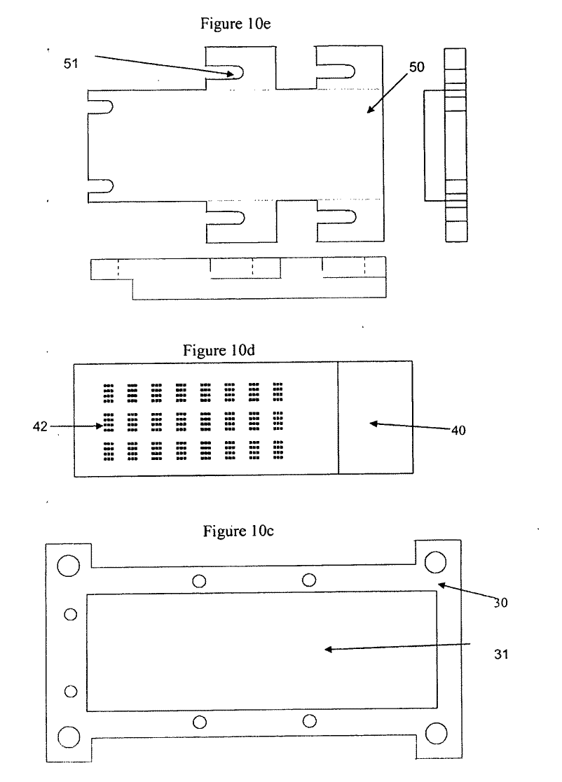

FIGS. 10a-10e show a top view of various layers of one embodiment of a device according to the invention which is capable of producing both a thermal and a chemical gradient.

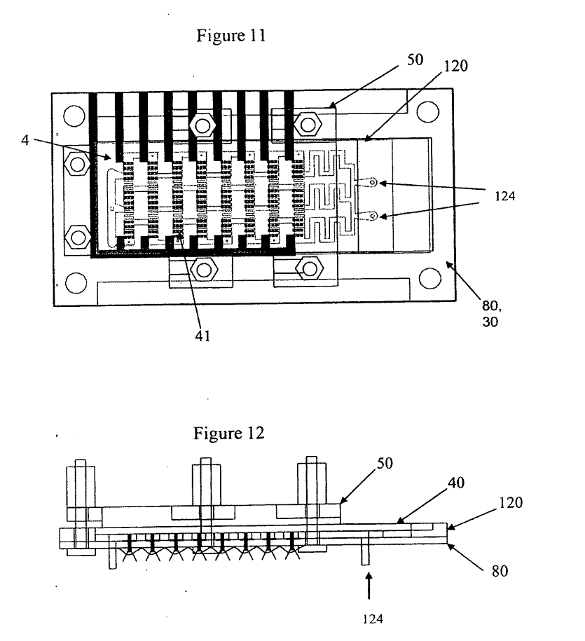

FIG. 11 shows a top view of one embodiment of the assembled device according to the present invention, which essentially comprises the parts shown in FIGS. 10a-10e, combined with a solid support.

FIG. 12 shows a side view of the embodiment shown in FIG. 11.

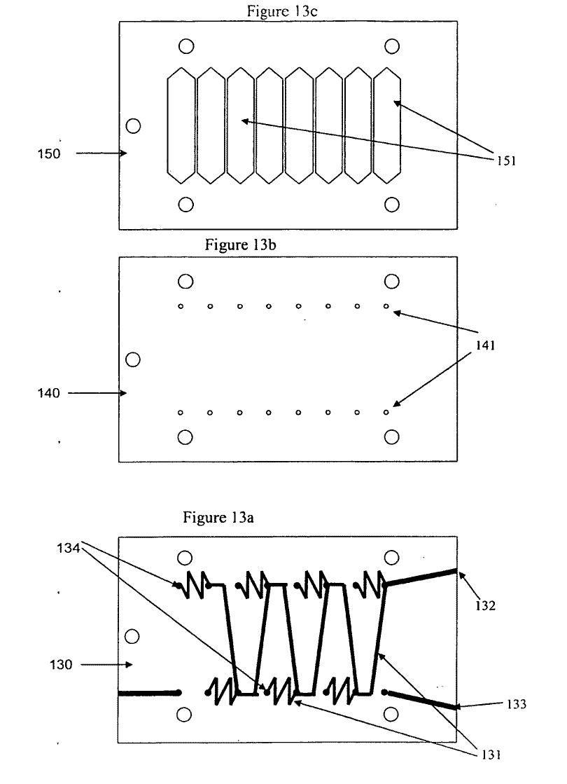

FIG. 13a-13c show a top view of various layers of yet another embodiment of a device according to the invention which is capable of producing a chemical gradient.

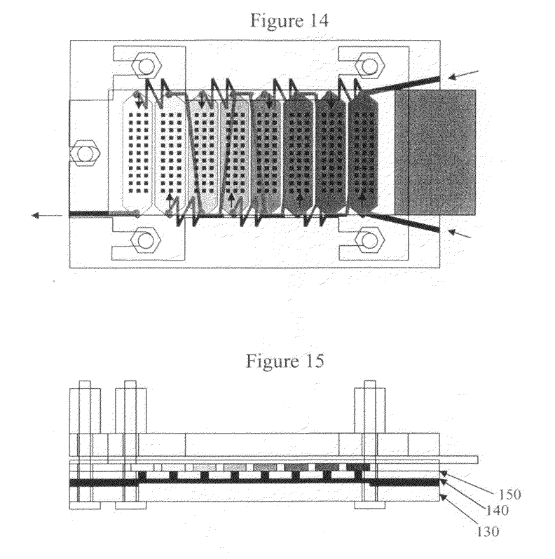

FIG. 14 shows a top view of one embodiment of the assembled device according to the present invention, which essentially comprises the parts shown in FIGS. 13a-13c, combined with a solid support.

FIG. 15 shows a side view of the embodiment shown in FIG. 14.

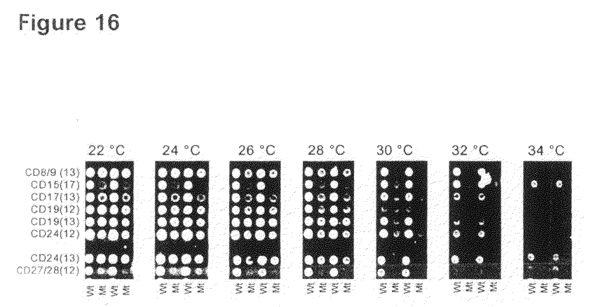

FIG. 16 shows images of fluorescently labeled DNA microarray after stringent washing at different temperatures provided by the device.

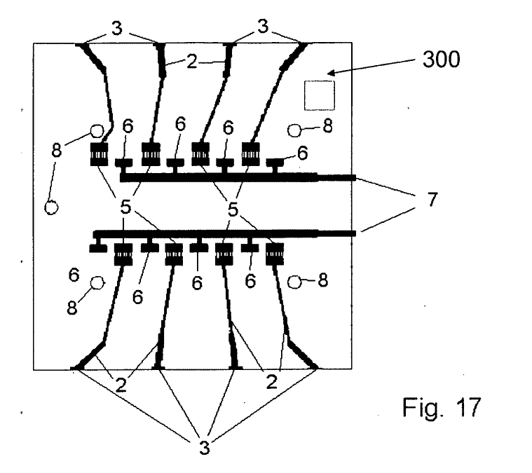

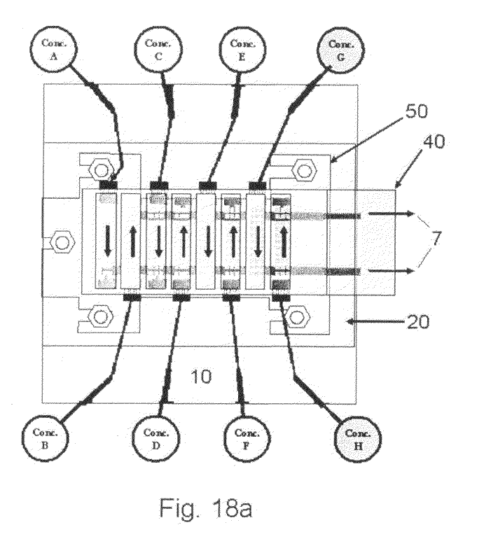

FIG. 17 shows a first layer of a microchannel device fed with different concentrations of washing fluids by gravity.

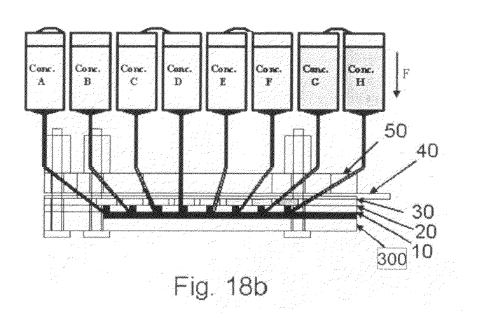

FIGS. 18a and 18b show a top view and a side view of the embodiment of a first layer of FIG. 17 in assembled form.

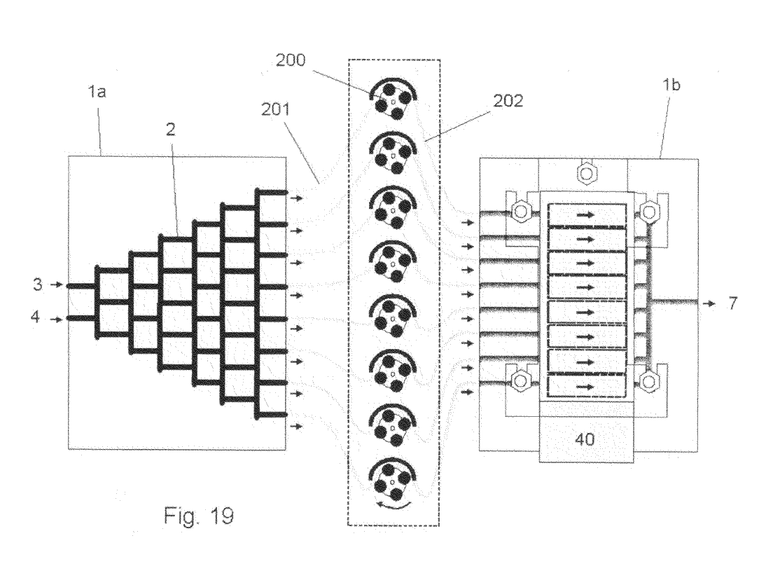

FIG. 19 shows an embodiment in which the first and the second layer of the base are split in two parts provided with pumps for transport of fluid from one part to the other.

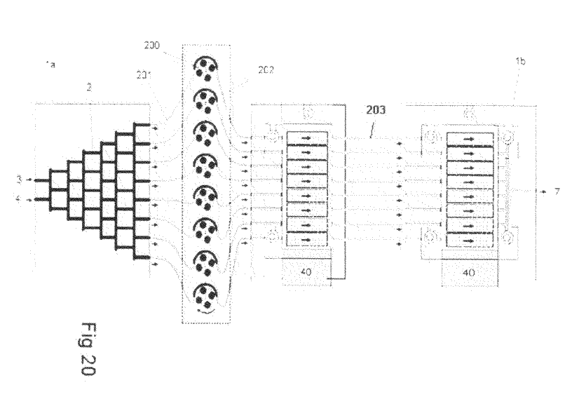

FIG. 20 shows an embodiment in which one or more solid supports can be inserted into a device by serially coupling several bases with washing chambers by tubes or microfluidic channels

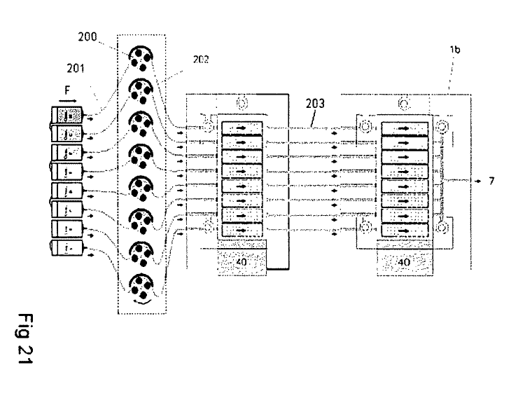

FIG. 21 shows an embodiment in which the different chemical conditions are created at different positions of the solid support by a flow of pre-mixed liquids.

FIGS. 22a and 22b show genotyping using a detection method with limited dynamic range such as absorbance measurements of alkaline phosphatase precipitations reaction on DNA arrays.

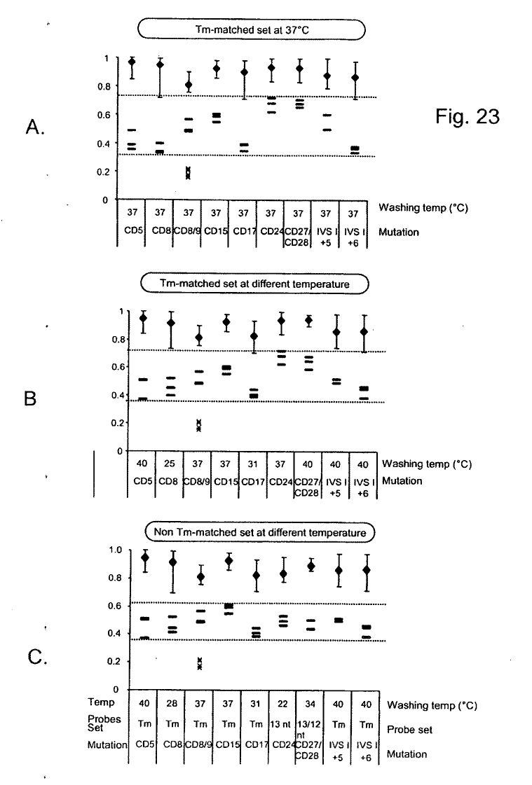

FIG. 23 show the effect of freely choosing probes and assay condition as compared with a temperature matched probe set at one temperature.

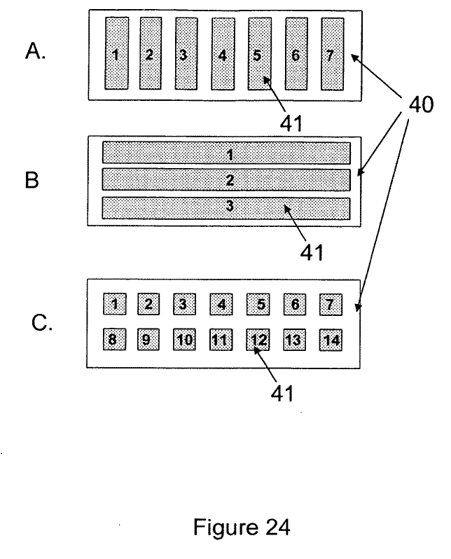

FIG. 24 shows possible layouts for where washing of a solid support is performed in the device.

FIG. 25 device combing a chemical gradient and different steady temperatures on each solid support in the series of connected bases.

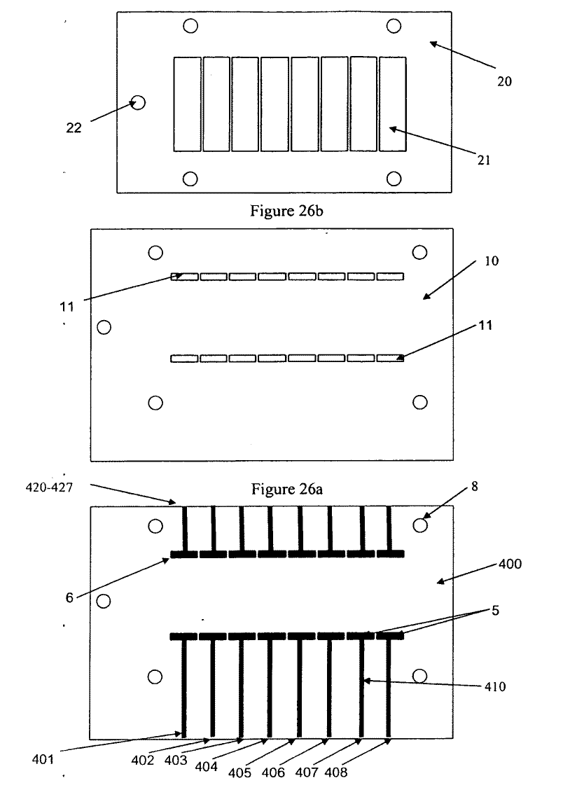

FIGS. 26a-26c shows a top view of various layers of one embodiment of a device according to the invention which is capable of leading fluids over different areas of a solid support.

The device can be made out of various numbers of layers, dependent on the fabrication method and intended function of the device.

FIG. 1a shows the first layer of a device which is capable of producing a chemical gradient. The first layer 1 has a microfluidic mixing tree 2 comprising a system of microchannels which microchannels are open towards the surface turned towards the second layer 10 during use. The microfluidic mixing tree 2 can mix two solutions in order to produce a chemical gradient. There are two inlets to the microfluidic mixing tree 2, a first inlet 3 for one solution, for example a salt solution, and a second inlet 4 for a diluent, for example water. On the other side of the microfluidic mixing tree 2 there are several exit points 5 to the chambers. The exit points can expel various concentrations of fluids produced in the microfluidic mixing tree 2, where each exit point expels different concentrations of the solution entered in the first inlet 3. Between one end of the exit point array to the other end of the exit point array there is provided a chemical gradient. The first layer 1 also has included therein a receiving structure 6 for receiving a fluid from chambers. The chambers will be described in relation to FIG. 1c. These structures are located essentially parallel to the exit points 5 of the microfluidic mixing tree 2. The fluid entering the receiving structure is discharged through an outlet 7. For facilitating the assembly of the device the first layer can have structures 8 for securing optimum assembly of the device, for example a protruding part which fits into respective holes in the other layers of the device. The structure 8 can also be a hole for receiving an external component which provides the same function. In the embodiment shown in FIGS. 1a-1f the alignment structure 8 is a hole for receiving an external screw.

FIG. 1b shows a second layer 10 which has essentially the same size as the first layer 1; said second layer 10 is having apertures 11 which correspond essentially to the exit points and the receiving structure in the first layer 1 shown in FIG. 1a. The second layer 10 closes the microchannels in the first layer 1 to form a closed system and the apertures 11 direct fluid from the first layer 1 to a third layer 20.

FIG. 1c shows the third layer 20 which functions as a sealing gasket (sealer). The layer has alignment holes 22 for securing and facilitating the assembly of the device. This third layer 20 also provides the chambers 21 when it is combined with the second layer shown in FIG. 1b and the solid support 40. The third layer 20 can be made of an elastic material forming a tight hermetic seal between the second layer 10 and the solid support when the solid support is inserted in the device and mechanical pressure is applied by a clamping structure. Clamping structures are shown in FIG. 1f. Alternatively, a reversible adhesive can be used on the surface of the third layer 20 to form a seal without the clamping means.

FIG. 1d shows a fourth layer 30 of the device. This fourth layer 30 defines which solid support can be inserted in the device by providing a structure 31 into which the solid support needs to fit. This fourth layer 30 can be exchanged by a layer with a different structure in order to make it possible to insert different solid structures into the same device. This layer also helps to automatically align capture structures on the solid support towards the corresponding chambers.

FIG. 1e shows an example of a solid support 40 onto which chemical entities 41 are attached. The solid support 40 is not actually a part of the device, but should correspond to the fourth layer 30 and is inserted during use. The solid support 40 in this case is a glass slide, which fits into the structure in the fourth layer 30. The solid support 40 is inserted into the device with the side of the support 40 where the chemical entities are attached facing downwards, i.e. facing the four layers 1, 10, 20, 30 of the device which form the base. As can be seen the solid support 40 in this case has an array 42 of chemical entities 41 attached to the surface (black squares) and furthermore being placed on the surface in such a way that the chemical entities 41 will be located in a chamber 21 when the solid support 40 is inserted in the device. The chambers 21 were described in relation to FIG. 1c.

FIG. 1f shows a lid 50 which can be used to facilitate the pressing of the solid support 40 towards the device, in order to secure proper sealing so no fluid can leak from the device. The lid 50 can have such a form that it receives a clamping means clamping the solid support to the device. One embodiment of such a clamping means is a nut and bolt, but it can also be any type of fastener e.g. an elastic tape, a clasp, a vice, etc. In the embodiment shown in FIGS. 1a-1f the clamping means is a screw which is inserted through a hole in the first layer and following through holes in the other layers, finally protruding from the device in the fourth layer. This is also discussed in relation to FIG. 1a. To each nut a corresponding bolt may be applied and fastened, such that the solid support and the device are pressed together.

FIG. 2 shows a top view of the layers 1, 10, 20, 30, 50 and the solid support 40 described in FIGS. 1a-1f assembled thereby forming the device. In this figure the same reference numbers as in relation to FIGS. 1a-1f are used.

FIG. 3 shows a side view of the layers described in FIGS. 1a-1f assembled thereby forming the device. In this figure the same reference numbers as in relation to FIGS. 1a-1f are used. Here it is clear how the layers are stacked on top of each other, forming the device. In some cases two or more layers can be combined in only one layer, for example the fourth layer 30 can be made from the same material as and be a part of the third layer 20.

FIGS. 4a-4g show a second embodiment of the invention where the second, the third and the fourth layers 10, 20, 30, the solid support 40 and the lid 50 are essentially the same as the similar layers of the embodiment of the device shown in FIGS. 1c-1f. Accordingly, it is the layers shown in FIGS. 4a and 4b which differ.

FIG. 4a shows a lowest layer 60 of the device, which in this case lies below a first layer 70, for performing a chemical gradient. In the lowest layer 60 a microfluidic mixing tree 61 is formed, which provides mixing of two solutions for providing a chemical gradient. There are two inlets to the microfluidic mixing tree 61, a first inlet 62 for one solution for example a salt solution and a second inlet 63 for a diluent, for example water. On the other side of the microfluidic mixing tree 61 are several exit points 64 for the mixed fluids, i.e. an exit point array, where in each exit point different concentrations of the solution entered in the first inlet 62 are provided and a chemical gradient is provided between the two ends of the exit point array. For facilitating the assembly of the device the lowest layer 60 can have structures 65 for securing optimum assembly of the device, for example a hole or a protruding part which fits into respective holes in the other layers of the device. In the embodiment shown in FIGS. 4a-4g the alignment structure is a screw and bolt.

FIG. 4b shows a first layer 70 providing a fluid connection between the exit points in the lowest layer 60 and the chambers 21 which are formed in a higher layer 20. The chambers 21 will be discussed in relation to FIG. 4c. The first layer 70 preferably has channels 71 providing fluid communication from one surface of the layer 70 to the other surface. As said above, this layer 70 provides a fluid connection between the exit points 64 in the lowest layer 60 and the chambers 21 in the subsequent layer 20, with a network of channels. The number of “inlets” and “outlets” in this layer normally corresponds to the number of chambers 21 in the subsequent layer 20. The first layer 70 shown in FIG. 4b has channels 71 providing connection between the exit points 64 on one axis and provides access from each of those to chambers 21 being arranged on an axis perpendicular thereto. However, it is also possible that the lowest layer 60 provides fluid connection between exit points and chambers being arranged on the same axis.

FIG. 5 shows a top view of the layers 60, 70, 10, 20, 30, 50 described in FIGS. 4a-4g assembled and thereby forming the device having the solid support 40 inserted. In this figure the same reference numbers as in relation to FIGS. 4a-4g are used.

FIG. 6 shows a side view of the layers described in FIGS. 4a-4g that are assembled to form the device. In this figure the same reference numbers as in relation to FIGS. 4a-4g are used. Here it is shown how the layers are stacked on top of each other, forming the device. In some cases two or more layers can be combined in only one layer, for example the fourth layer 30 can be made from the same material as, and be a part of, the third layer 20.

FIGS. 7a-7e show a third embodiment of the device. FIG. 7a shows the first layer 80 of an embodiment of a device which layer provides the device with the possibility of creating a thermal gradient. The first layer 80 comprise wires 81, 82, and in a first area, corresponding to the area where the chambers 91 are formed in a second layer 90, the wires 81 have a relatively small diameter and are formed in a zig-zag structure. Accordingly, this part of the wire 81 can provide local heating of fluid in each chamber 91. In a second area the wires 82, which in this embodiment are placed mainly outside the area corresponding to where the chambers 91 are formed in a second layer 90, have a larger diameter (cross section) and accordingly do not produce as much heat when electric current is passed through them. Each zig-zag structure 81 has its own connection via the wire parts 82 to an electronic control circuit or an energy source, to facilitate regulation of the temperature in each chamber easily. For assisting the assembly of the device, the first layer 80 can have structures 83 similar to the ones describe in relation to FIG. 1a. In the embodiment shown in FIGS. 7a-7e the alignment structure 83 is an external screw. The first layer 80 furthermore has two holes 84 for providing access to the inlet 92 and outlet 93 holes in the microchannels described in FIG. 7b.

FIG. 7b shows a second layer 90, which functions as a sealing gasket. This second layer 90 also provides the chambers 91 when it is combined with the first layer 80 shown in FIG. 7a and the solid support 40 shown in FIG. 7d. The second layer 90 can be made of an elastic material forming a tight hermetic seal between the second layer 90 and the solid support 40 when the solid support 40 is inserted in the device. In this case the chambers 91 are not isolated from each other but rather defined as areas in a continuous microchannel 94. However, the chambers 91 can also easily be separated from each other in which case each chamber 91 has an inlet and an outlet for fluid. The channel 94 can facilitate the stringent washing of the chemical entities on a solid support. The microchannel has an inlet 92 and an outlet 93 for the fluid. In the embodiment shown in this figure the fluid can flow into the colder area of the gradient and run over to the hotter area of the gradient. This layer has furthermore holes 95 for receiving thermal sensors.

FIG. 7c shows a third layer 100 of the device. This layer 100 defines which solid support 40 can be inserted in the device by providing a structure 101 into which the solid support 40 needs to fit. This layer 100 can be exchanged with another layer with a different structure in order to make it possible to insert different solid supports 40 into the same device. As mentioned in relation to FIG. 7a the third layer 100 also has holes 102 for receiving an alignment structure.

FIG. 7d shows a solid support 40 onto which chemical entities are attached. The solid support 40 is not actually a part of the device, but should be inserted during use. The solid support 40 in this case is the same solid support 40 as described in relation to FIG. 1e, i.e. a glass slide, which fits into the space created by the structure of the third layer 100 of FIG. 7c.

FIG. 7e shows the same lid 50 as is described in relation to FIG. 1f, i.e. a lid 50 which can be used to facilitate the pressing of the solid support 40 in contact with the device, in order to secure proper sealing so no fluid can leak from the device.

FIGS. 7a-7b contain holes for facilitating temperature sensors (thermistors). One temperature sensor per chamber 91 is used close to the end towards the outflow point for fluid of each chamber 91.

FIG. 8 shows a top view of the layers 80, 90, 100, 50 described in FIGS. 7a-7e assembled thereby forming the device having the solid support 40 inserted. In this figure the same reference numbers as in relation to FIGS. 7a-7e are used.

FIG. 9 shows a side view of the layers 80, 90, 100, 50 described in FIGS. 7a-7e assembled thereby forming the device having the solid support 40 inserted. In this figure the same reference numbers as in relation to FIGS. 7a-7e are used. Here it is clear how the layers are stacked on top of each other thereby forming the device. In some cases two or more layers can be combined in only one layer. This figure also shows how temperature sensors 112 are inserted to the chambers 91 through the holes in layers 70 and 80, and fixed by the elastic glue 111. Such configuration allows small displacement of the temperature sensors 112 without breaking the seal of the chamber when the clamping pressure is applied to the solid support 40.

FIG. 10a shows the first layer 80 of a device providing a combined chemical and a thermal gradient. The first layer 80 has the same components as described in relation to FIG. 7a, which provides a resistive component for providing a thermal gradient.

FIG. 10b shows a second layer 120 which functions as a sealing gasket. This second layer 120 also provides chambers 121, 122 having varying chemical concentrations 121 and having varying thermal conditions 122 when the second layer 120 is combined with the first layer 80 shown in FIG. 10a and the solid support 40 shown in FIG. 10d. The second layer 120 is provides microchannels 126 for the fluid similar to the layer shown in FIG. 7b but there are several differences. The second layer 120 also has a microfluidic mixing tree 123 providing a chemical gradient as shown in FIG. 4a, but on a smaller scale. The microfluidic mixing tree 123 has two inlets 124 and three outlets 125, wherefrom fluids having different chemical concentrations are expelled, each of said outlets 125 being connected to a microchannel 126. Each of these microchannels 126 functions essentially as the one described in relation to FIG. 7b. Accordingly, there is provided a matrix of two parameters, i.e. three concentrations and eight temperatures.

FIGS. 10c-10e show essentially the same layers of the device as are shown in FIGS. 7c-7e.

FIG. 11 shows a top view of the layers described in FIGS. 10a-10e assembled thereby forming the device. In this figure the same reference numbers as in relation to FIGS. 10a-10e are used.

FIG. 12 shows a side view of the layers described in FIGS. 10a-10e assembled thereby forming the device. In this figure the same reference numbers as in relation to FIGS. 10a-10e are used. Here it is clear how the layers are stacked on top of each other, forming the device. This figure also shows how temperature sensors are inserted to the chambers 121, 122 through the holes in layers 120 and 40, and fixed by elastic glue. Such configuration allows small displacement of the temperature sensors without breaking the seal of the chamber when the clamping pressure is applied to the solid support.

The device shown in FIGS. 13a-13c show essentially the same layers of the device as are shown in FIGS. 1a-1c. The other layers are not shown as they are essentially the same as the layers in FIG. 1d-1f.

FIG. 13a shows a lowest layer 130, having a different type of a microfluidic mixing tree 131 for performing a chemical gradient. There are two inlets 132, 133 to the microfluidic mixing tree 131, a first inlet 132 for one solution for example a salt solution and a second inlet 133 for a diluent, for example water. There are several exit points 134 of the microfluidic mixing tree for the entered fluids on various places of the mixing tree 131, where different concentrations of the solution entered in the first inlet 132 are provided in each exit point. Other structures of this layer are the same as described in other embodiments.

FIG. 13b shows a first layer 140 for providing a fluid connection between the exit points 134 in the lowest layer 130 and the chambers 151 which are formed in subsequent layers. The chambers 151 will be discussed in relation to FIG. 13c. The first layer 140 preferably has microchannels 141 providing fluid communication from one surface of the layer to the other surface. As said above, this layer provides a fluid connection between the exit points 134 in the lowest layer 130 to the chambers 151 in subsequent layers, with a network of microchannels 141. The number of “inlets” and “outlets” in this layer corresponds to the number of chambers in a subsequent layer.

FIG. 13c shows a second layer 150 of a sealer forming the chambers 151. This figure shows a possible different form of the chambers, but could also be formed as described in relation to the embodiments in other figures.

FIG. 14 shows a top view of the layers 130, 140, 150 described in FIGS. 13a-13c assembled and thereby forming the device having a solid support 40 inserted. In this figure the same reference numbers as in relation to FIGS. 13a-13c are used.

FIG. 15 shows a side view of the layers described in FIGS. 13a-13c that are assembled to form the device. In this figure the same reference numbers as in relation to FIGS. 13a-13c are used. Here it is shown how the layers 130, 140, 150 are stacked on top of each other together with the layers which are the same as in the other embodiments, forming the device in a ready-to-use state. In some cases two or more layers can be combined in only one layer, for example the fourth layer can be made from the same material as, and be a part of, the third layer.

FIG. 16 shows the effects on genotyping using the device shown in FIG. 13. The DNA probes was deposited onto agarose film coated glass slides according to the procedure described in [1] [Dufva, M.; Petronis, S.; Bjerremann Jensen, L.; Krag C.; Christensen, C.; “Characterization of an inexpensive, non-toxic and highly sensitive microarray substrate”, Biotechniques, 37 (2004) 286-296] using probes toward specific mutations occurring in the b-globin gene. The probes are tagged with a “TC tag” (as described [2] [Dufva, M.; Petersen, J.; Stoltenborg, M.; Birgens, H.; Christensen, C. B.; “Detection of mutations using microarrays of poly(C)10-poly(T)10 modified DNA probes immobilized on agarose films”, Anal Biochem, 352 (2006) 188-97]) to increase the amount of functional probes on the surface. Thus “CD8/9” for instance in the graph denotes a probe set that analysis for a specific mutation occurring at codons 8 and 9. A short fragment on the be-globin gene was amplified by PCR using primers described in table 1 (se below). Single stranded DNA was generated by linear amplification of the PCR products using only reverse primers in the reaction. The reverse primer was fluorescently labeled to be able to subsequently visualise hybridisations to arrays using a fluorescent scanner. The single stranded fluorescently labeled DNA was hybridised to the microarray by applying 120 μL hybridisation solution (60 μL 10×SSC (saline-sodium citrate) +1% SDS (sodium dodecyl sulphate) and 60 μL single stranded DNA). The hybridisation solution was spread over the slide by applying a coverslip large enough to cover the entire slide. In such the microarray was hybridised in a humid chamber for 2 h at 37° . The slide was rapidly washed using a low stringent washing buffer such as 5×SSC and the slide was loaded in the apparatus described in FIG. 13. A flow of 0.3×SSC+0.1% SDS was applied in the formed channels using a pump. The computer controlled heating of the individual structures is started and the slide is washed for a defined time period of 30 minutes. Other incubations periods can be used if desired. The slide is subsequently dried and scanned in a fluorescent scanner.

TABLE 1
Probes arrayed in the example given in FIG. 16.
The number in parenthesis denoted the length
of the specific sequence.

| | |
|---|---|
| CD8/9 mt | TTTTTTTTTTCCCCCCCCCC GAG AAG G TCT |
| | GCC |
| CD8/9 wt | TTTTTTTTTTCCCCCCCCCC G GAG AAG TCT |
| | GCC |
| CD15 mt | TTTTTTTTTTCCCCCCCCCC TGC CCT GTA |
| | GGG CAA GG |
| CD15 wt | TTTTTTTTTTCCCCCCCCCC TGC CCT GTG |
| | GGG CAA GG |
| CD17 mt | TTTTTTTTTTCCCCCCCCCC GTG GGG CTA |
| | GGT G |
| CD17 wt | TTTTTTTTTTCCCCCCCCCC GTG GGG CAA |
| | GGT G |
| CD19(13) mt | TTTTTTTTTTCCCCCCCCCC AGG TGG ACG |
| | TGG A |
| CD19(13) wt | TTTTTTTTTTCCCCCCCCCC AGG TGA ACG |
| | TGG A |
| CD19(12) mt | TTTTTTTTTTCCCCCCCCCC AGG TGG ACG |
| | TGG |
| CD19(12) wt | TTTTTTTTTTCCCCCCCCCC AGG TGA ACG |
| | TGG |
| CD24(13) mt | TTTTTTTTTTCCCCCCCCCC GTT GGA GGT |
| | GAG G |
| CD24(13) wt | TTTTTTTTTTCCCCCCCCCC GTT GGT GGT |
| | GAG G |
| CD24(12) mt | TTTTTTTTTTCCCCCCCCCC GTT GGA GGT |
| | GAG |
| CD24(12) wt | TTTTTTTTTTCCCCCCCCCC GTT GGT GGT |
| | GAG |
| CD27-28 mt | TTTTTTTTTTCCCCCCCCCC GAG GCC C CTG |
| | GGC |
| CD27-28 wt | TTTTTTTTTTCCCCCCCCCC GAG GCC CTG |
| | GGC A |

FIG. 17 shows the first layer of a device which when assembled (FIG. 18) transport liquid by gravity. The device is capable of producing a chemical gradient or different chemical conditions in different washing chambers 21. The first layer 300 of this embodiment comprises a total of eight entering channels 2 for pre-mixed washing fluid. The pre-mixed washing fluid is kept in reservoirs positioned above the washing chambers in order to provide a steady fluid pressure due to gravity on the discharge end of the entering channel 2. Each entering channel 2 has an inlet 3 which is connected to the reservoir of pre-mixed fluid. As the first layer 300 has eight entering channels 2 it is possible to pre-mix eight different washing fluids having e.g. a varying content of a salt solution and then transferring the washing fluids to the chambers through the entering channels 2. Like the embodiment of FIG. 1a the channels are open towards the surface turned towards a second layer during use. Each entering channel 2 has an exit point 5 to a chamber, thus between the eight exit points provided different chemical conditions or a chemical gradient is created. The first layer 300 also includes a receiving structure 6 for receiving a fluid from the chambers. Similar chambers 21 were described in relation to FIG. 1c. The fluid entering the receiving structure 6 is discharged through an outlet 7. This first layer 300 also has structures 8 for securing optimum assembly of the device, in this embodiment the structure 8 is a series of holes for receiving an external screw. This embodiment also comprise a second layer corresponding to the layer 10 shown in FIG. 1b, closing the channels in the first layer 1 to form a closed system and apertures direct fluid from the first layer 1 to a third layer corresponding to the layer 20 shown in FIG. 1c. Both in the embodiment shown in FIG. 1 and in FIG. 7 the third layer 20 functions as a sealing gasket (sealer) forming a tight hermetic seal between the second layer 10 and the solid support when the solid support is inserted in the device. Further the device according to the embodiment of FIG. 7 comprises a fourth layer corresponding to the layer 30 shown in FIG. 1d defining which solid support can be inserted in the device by providing a structure 31 into which the solid support needs to fit, a solid support corresponding to the support 40 in FIG. 1e, and a lid corresponding to the lid 50 shown in FIG. 1f.

FIG. 18a shows the embodiment of FIG. 17 seen from above where all the layers are assembled. The entering channels 2 of the first layer 300 are joined to eight reservoirs of solutions with concentration A, B, C, D, E, F, G and H. The reservoirs are raised above the level of the device.

FIG. 18b shows the embodiment of FIG. 18a from a side view.

FIG. 19 shows a top view of an embodiment of a split chip design, where the original chip comprising the first layer 1 shown in FIG. 1a is split in two i.e. the layer comprises a first part 1a containing a microfluidic mixing tree and a second part 1b comprising washing chambers attached to microarray slide.

8-channel peristaltic pump 200 is placed in between these two parts 1a and 1b comprising a soft tubing 201 leading fluid to the pump and a soft tubing 202 leading fluid from the pump 200 to the solid support mounted on the base 40, and assure equal suction of liquids through all channels 2 in the mixing tree. Including the pumps 200 thus causes a very reliable concentration or chemical gradient as the flow through the mixing tree is more predictable. Furthermore, bubble traps can be placed in between the peristaltic pump 200 and the chambers in the base 40, to assure high quality washing of the solid support (this feature is not shown in the fig).

FIG. 20 shows an embodiment where two solid supports similar to the one used in FIG. 19 are inserted into two devices. The chambers 21 which are defined by the third layer as shown in FIGS. 1c and 4d are then serially coupled by tubes or microfluidic channels 203. If expedient more than two supports could be serially joined.

FIG. 21 shows an embodiment having serially coupled chambers 21 in which embodiment the different chemical conditions are created on the different parts of the solid support by pumping of pre-mixed liquids using e.g. a peristaltic pump.

FIGS. 22a and 22b shows genotyping using detection methods with limited dynamic range such as absorbance measurements of alkaline phosphatase precipitations reaction on DNA arrays. In FIG. 22a the colorimetric and fluorescence method is compared for single stringency (e.g 0.3×SSC+0.5 SDS, 28° C.). In FIG. 22b the result of using the colorimetric method at the three different stringencies (as modulated by temperature in this case but in other cases chemically) are shown. It turns up that at varying stringency the colorimetric performs just as well as the fluorescence method performed at a single stringency.

FIG. 23 shows the effect on genotyping using a gradient device and probes that are working at different conditions. In this case a thermal gradient is shown but the described effects are also seen with a chemical gradient. The performance of the gradient generating device on array assay was tested using a set of Tm matched probes (denoted as “Tm” in the figures) and a set of non-Tm matched shorter probes (denoted with the length of the probe (13 nt or 13/12 nt)) for mutation detection in the beta-globin gene. The Tm matched set of probes was designed using thermodynamic models. The probes included in this set varied in length between 15 nt and 21 nt and had a theoretical Tm of 50° C. A short probe set with non Tm matched probes was also constructed, where the only requirements was that the probes are as short as possible but still give signals in hybridization reactions. These probes were mainly 12-17 nt long and had melting temperatures in the range 35-57° C. Both probe sets where printed in eight identical subarrays on agarose film coated slides. The slides were hybridized with amplified DNA samples from 29 beta-thalassemia carriers, previously genotyped using Sanger sequencing and two controls with no mutations. The hybridized slides were mounted in the device and the subarrays were washed for 30 minutes with temperatures ranging from 22-43° C., with three degrees increment. After quantification of the spots i.e. the chemical entities, a ratio of the signals (Wt/wt+mt) for each mutation and condition was calculated. The ratios were organized into classes based on sanger squencing. Hence all the homozygote wildtypes are denoted with a diamond (error bars represents min and max values of the respective homozygote wildtype. The heterozygotes for each mutation are denoted with a dash and the homozygote mutated is denoted with a cross. A. genotyping results with a melting temperature matched set at one temperature. The perfect match probes were chosen using thermodynamic models to predict the melting temperature. Theoretically, the probes had a melting temperature of about 50° C. Using the device with upper and lower range of temperature (stringency) of 22 and 40° C., the acceptable condition for all probes was 37° C. However is should be noted that by washing at this stringency some homozygote values overlapped with wildtype values of the ratio which is not desirable. It is primarily the heterozygotes that vary. B. Genotyping results of a Tm matched set at different temperatures. Optimal working condition is defined as the condition where the heterozygotes are as close to 0.5 as possible and the homozygote wild types are as close to 1 as possible and homozygote mutants are as close to 0 as possible. The thermodynamically predicted probe set functioned optimally between 25° C. and 40° C. Applying optimal temperature for each probe pair in a Tm matched set, led both to slightly lower variance of the heterozygotes and to lower error frequency in genotyping. For instance the error frequency of the probes towards CD27/CD28 of the Tm matched set was reduced from 1 in 10¹² (at 37° C.) to 1 in 10¹⁸ when washing at optimal temperature of 40° C. However, the probe for CD27/28 still gives high ratio for a hetrozygote which is less optimal (FIG. 3B). C. Genotyping using a gradient and probes with widely different optimal working conditions. In this assay probes was chosen to (i) give signals and (ii) to function optimally in the assay. The probes were not chosen to work at the same temperature. Optimal working condition is defined as the condition where the heterozygotes is as close to 0.5 as possible and the homozygote wild type is as close to 1 as possible and homozygote mutants as close to 0 as possible. It is clear that the variation of heterozygotes could almost be halved if the fluidics device creating a temperature gradient over the array was used in combination with probes with different working optimum. A multi-condition (temperature or chemical) assay can thus be used for simple calling of genotypes where homozygote mutated ranged between 0.7 and 1, heterozygotes between 0.37 and 0.61 and homozygote mutated less than 0.25 (FIG. 3C). Without the gradient provided for in the device it would be very difficult for instance to include analysis of CD24 in the same assay.

FIG. 24 shows possible locations of subarrays of chemical entities (41) on an asymmetric solid support (40). The chambers (21) of the device should be located in such a way that the subarrays can be subjected to different conditions. The base such as the embodiment shown in FIG. 26 must thus be modified accordingly to provide a chamber (21) for each subarrays. The layout of the chambers (21) depends on the basic form of the solid support (40), and the number of desired condition that is going to be investigated. It may also depend on the way a microarray is fabricated. Accordingly, the chambers (21) defined by the base (e.g 400, 10, 20, 30) must be changed to fit these requirements. For instance, Agilent produces slides with eight identical subarrays organized as four column and two rows (similar to the configuration of 24C) and a base (400, 10, 20, 30) must be changed to fit the arrays within chambers. Basically a new layout of chambers and channels defined in (400) and (10) must be employed. The most common format of the solid support (40) which is the microscope slide is shown but the invention is not limited to be applied to microscope slides format or particular fabrication methods. The invention can handle basically all possible formats of solid support and number of areas that can be exposed to different conditions. However, the device is preferable working with chambers that are minimum 3×3 mm to minimize problems with aligning the slide with subarrays to the chambers (21). A. microscope slide is normally provided with 7 chambers but the number of chambers is not limited to 7. A device could for instance have 16 such chambers where each chamber is 3.5 mm wide and 20 mm long and with a 1 mm wall between each chamber. B. The chamber can also be organised to be on the long side of an asymmetric solid support (40). However, fewer chambers could then be fitted on such solid supports. The benefit is that very large chambers (21) can be utilized, allowing large arrays to be processed. C. Other organization of chambers. Some fabrication devices prefer to print subarrays as two rows and many columns.

FIG. 25 shows the combination of a chemical gradient and thermal gradient where the chemical gradient is created on each slide and the respective solid support (40) is treated with different temperatures. In order to span a large range of assay condition, it may be required to investigate chemical gradients in combination with a temperature gradient. One solution is to serially or parallel couple several e.g. 6-12 bases (400, 10, 20, 30) thereby creating chemical gradients over a series of chambers on each slide. The temperature of a slide could be regulated using a hot plate (500) or similar heating device (resistive elements, Peltier elements, tempered air or tempered liquids (water, oils etc). Because we can couple 6 to 12 bases (400, 10, 20, 30) to the same pumping devices, we can investigate the chemical gradient at up to twelve different temperatures provided that each temperature block (500) can be controlled individually. The figure shows a device that can investigate eight different chemical conditions at two different temperatures, one temperature for each slide.

FIG. 26 shows the first layer of a device which is capable of flowing liquids over a solid support (40). The first layer 400 has a system of channels (410) which are open towards the surface turned towards the second layer 10 during use. After closing of the layers 400 and 10, the formed channels (401-408) can be connected to various sources of externally created chemical gradients. For instance the inlet 401 is operatively connected to a beaker or flask or a vessel containing a solution, while inlet 402-408 is operatively connected to other flasks or beakers or vessels. Inlets 401 and 408 can also be connected to outlets of a tree structure providing automatic generation of a gradient (FIG. 19 and FIG. 20). The channels (401-408) are at the ends connected to one of several exit points 5 leading fluid to the chambers. The exit points can expel various concentrations of fluids from external sources where each exit point expels different concentrations of a solution entered in the inlet 401-408. The first layer 400 also has included therein a receiving structure 6 for receiving a fluid from chambers. The chambers will be described in relation to FIG. 1c. These structures are located essentially parallel to the exit points 5. The fluid entering the receiving structure is discharged through an outlet 420-427. For facilitating the assembly of the device the first layer can have structures 8 for securing optimum assembly of the device, for example a protruding part which fits into respective holes in the other layers of the device. The structure 8 can also be a hole for receiving an external component which provides the same function. In the embodiment shown in FIGS. 1a-1f the alignment structure 8 is a hole for receiving an external screw.

FIG. 26b shows a second layer 10 which has essentially the same size as the first layer 400; said second layer 10 is having apertures 11 which correspond essentially to the exit points and the receiving structure in the first layer 400 shown in FIG. 26a. The second layer 10 closes the microchannels in the first layer 400 to form a closed system and the apertures 11 direct fluid from the first layer 400 to a third layer 20.

FIG. 26c shows the third layer 20 which functions as a sealing gasket (sealer). The layer has alignment holes 22 for securing and facilitating the assembly of the device. This third layer 20 also provides the chambers 21 when it is combined with the second layer shown in FIG. 1b and the solid support. The third layer 20 can be made of an elastic material forming a tight hermetic seal between the second layer 10 and the solid support when the solid support is inserted in the device and mechanical pressure is applied by a clamping structure. Clamping structures are shown in FIG. 1f. Alternatively, a reversible adhesive can be used on the surface of the third layer 20 to form a seal without the clamping means. The parts or layers 400, 10, 20, 30 are connected as described in FIG. 1. The device is subsequently operatively connected to the solid support 40 by pressing the lid 50 towards the channels structures 400, 10, 20 in the same fashion as described in FIG. 1.

Generally the device is operated in the following manner:

The device is capable of forming a temperature gradient:

A clean device is provided with electric and fluid connections.

A solid support onto which chemical entities are attached is inserted in the device with the chemical entities facing the device and the chambers formed by the sealer.

The solid support and the device are clamped together providing a secure hermetic seal for the channels and chambers. Optionally the channel network can be washed at room temperature with a fluid (e.g. salt solution), which does not interact with the chemical entities at ambient temperature.

Thereafter the channel network and the chambers are filled with an assay fluid (e.g. buffer containing an analyte) so that the analyte can interact with the chemical entities attached to the solid support.

Then, a current is applied to the temperature regulators in the device thereby beginning the heating/cooling action and creating the different thermal assay conditions in each chamber.

After a predetermined assay time, or when the sensors have confirmed the desired temperature profile, the current may be switched off or controlled at a constant temperature and the microchannel network and chambers are thereafter washed by a neutral buffer flow. The flow removes unspecifically bound or dissociated chemical entities.

Thereafter the solid support is removed from the device, optionally washed again, dried, and the amount and position of remaining bound analyte is detected by appropriate methods. The detection of the signal is dependent on the type of assay performed.

The device is then washed and ready for reuse.

Alternatively, a solid support with attached chemical entities can be exposed to an assay fluid before mounting it to the device.

After a predetermined assay time, the solid support is inserted into a clean device provided with electric and fluid connections. The solid support and the device are clamped together providing a secure hermetic seal around the chambers.

The microchannel network and the chambers are filled and continuously flushed with a stringent washing fluid (e.g. salt solution).

A current is applied to the temperature-regulators in the device thereby beginning the heating/cooling action and creating different thermal washing conditions in each chamber. The flow removes the dissociated chemical entities, so that they do not bind back to the solid support.

After a predetermined time, or when the sensors have confirmed the desired temperature profile, the current may be switched off or controlled at a constant temperature.

The solid support is removed from the device, optionally washed again, dried, and the amount and position of remaining bound analyte is detected by appropriate methods. The detection of the signal is dependent on the type of assay performed.

The device is then washed and ready for reuse.

The device is capable of forming a chemical gradient:

A clean device is provided with fluid connections. A solid support onto which chemical entities are attached is inserted in the device with the chemical entities facing the device and the chambers formed by the sealer. The solid support and the device are clamped together providing a secure hermetic seal. The microchannel network can optionally be washed with a fluid (e.g. salt solution) which does not interact with the chemical entities. Purging can be done via one or both inlets and this can be done under pressure, by pumping, by capillary forces or by suction via outlet. Thereafter a fluid having a chemical concentration is transferred to the first inlet and a diluent (e.g. water) to the second inlet. After a predetermined time, for example when the chambers and chemical entities within have been flushed with each concentration, the channel network is purged with another fluid (e.g. the same fluid as used in the first purging step). The solid support is removed from the device and an amount and position of remaining bound analyte is detected by appropriate methods, dependent on the type of assay performed. The device is thereafter washed and ready for reuse. The profile of the gradient can be controlled by the pumping rates of the salt solution and water/diluent.

The device is capable of forming a temperature gradient and a chemical gradient:

A clean device is provided with electrical and fluid connections. A solid support onto which chemical entities are attached is inserted in the device with the chemical entities facing the device. The solid support and the device are clamped together providing a secure hermetic seal. Optionally the first step is to purge the channel network with a salt solution that does not interact with the chemical entities. Thereafter a solution having a chemical concentration is transferred to the first inlet and water to the second inlet. This can be done under pressure, by pumping, by capillary forces or by suction via outlet. After a predetermined time, when the chambers have been flushed with each concentration, the current is applied to the temperature regulator. Finally when the desired temperature profile is obtained the current is switched off and the channel network is allowed to cool down to ambient temperature. The channels are purged with salt concentration only. The solid support is removed from the device and an amount and position of remaining bound analyte is detected by appropriate methods, all dependent on the type of assay performed. The device is now washed and ready for reuse.

If necessary, the device can also be used as a hybridisation chamber.

### EXAMPLES

PCR amplified DNA from the β-globin region was hybridised to microarrays of probes (table 1) designed to detect specific mutation in the β-globin gene. The length of the specific part of the probes is denoted in parenthesis. After hybridisation, the slide was mounted into the multithermal device and washed at different temperature as indicated in FIG. 13 using the device. The result shows that some probe pairs worked best at different temperatures (FIG. 16). For example good discrimination between wild type and mutant probe hybridisation was observed for CD27/28, CD15, CD17 at 22° C. while for example CD24(13) resulted in maximum discrimination at 30° C. and CD19(13) at 28° C. If genotyping was made at 22° C., the DNA would be considered to be heterozygote for many position (like CD27/28 and CD19) demonstrating the strength of using multiple conditions for genotyping. The results show also that if a single condition is preferable to use, 28° C. give satisfactory results for most of the probes.

Therefore the experiments show that genotyping could be enhanced by washing at different temperatures. Similar results could be obtained by washing the slides in chemical gradient chip where room tempered washing solution with two different salt concentrations could be applied in the two inlets. For example 1×SSC (saline-sodium citrate)+0.1% SDS (sodium dodecyl sulphate) could be applied in inlet 1 and 1×SSC+0.1% SDS in inlet 2. The concentration range would be from 1×SSC to 0×SSC. Alternatively, the DNA hybrids could be broken at specific concentrations of NaOH, urea or any other denaturant.

The devices shown in FIG. 18a and b and FIG. 21 are particularly suitable when very precise and re-producible chemical gradients are needed. Because mixing is not dependent on flow but on the content of the reservoirs holding the pre-mixed buffers, each chamber of the array will be exposed to the a well-defined chemical solution. Devices in FIG. 18a and b and FIG. 21 are also particularly suitable when non-linear gradients are desired, which can be difficult to create by using device with integrated gradient generators such as the devices shown in FIGS. 5, 14, 15, 20.

### Colorimetric Method

When using the multiconditional washing it is possible to use a non-expensive colorimetric method to quantify the analyte-capture probe complexes. The multiconditional washing makes it possible to increase the dynamic range for the colorimetric method.

There are many colorimetric reaction or other detection methods which could potentially be used but which methods have a dynamic range which is to limited. One example is alkaline phosphatase (AP) chromagenic reaction utilising BCIP/NBT (Bromo-Chloro-Indolyl Phosphate/ NitroBlue Tetrazolium) as substrate). This method is widely used in immunochemistry and specifically implemented for microarrays detection by Petersen et al. (“Detection of mutations in the β-globin gene by colorimetric staining of DNA microarrays visualized by a flatbed scanner”, In Press, Uncorrected Proof, Available online 7 Sep. 2006; Petersen, J.; Stangegaard, M.; Birgens, H.; Dufva, M.). Briefly, AP mediated BCIP/NBT reaction on microarrays is done by first making arrays of oligonucleotides on suitable substrates such as agarose films (Dufva et al 2004 Biotechniques,37, 286-296). Target is prepared by enzymatic reactions in a way so that biotin or digoxigin is incorporated into the target molecules. After hybridization and stringency wash using gradient creating devices such as those described here, the arrays are incubated with streptavidin linked alkaline phosphatase. Finally, the arrays are incubated with BCIP/NBP for a defined period such as 10 minutes. The arrays can subsequently be digitized using a flatbed scanner, a CCD camera or a digital camera. There are many different similar enzymatic well known staining methods that can be used such as those based on horse radish peroxidise (HRP).

Other examples of detection methods having limited dynamic range and which can be used to detect microarray interactions is:
nano-gold particles staining as described by Taton, et al. (Taton, T. A., Mirkin, C. A., and Letsinger, R. L., “Scanometric DNA array detection with nanoparticle probes, Science”, 289 (2000) 1757-60) and Han et al. (Han, A., Dufva, M., Belleville, E., and Christensen, C., “Detection of analyte binding to microarrays using gold nano particles labels and a desktop scanner”, Lab. Chip., 3 (2003) 336-339),
silver enhanced gold particles staining as described by Taton, et al. and Alexandre, et al (Alexandre, I., Hamels, S., Dufour, S., Collet, J., Zammatteo, N., De Longueville, F., Gala, J. L., and Remade, J., Colorimetric silver detection of DNA microarrays, Anal Biochem, 295 (2001) 1-8),
carbon black particles as described by Lonnberg, M. et al. (Lonnberg, M., and Carlsson, J., Quantitative detection in the attomole range for immunochromatographic tests by means of a flatbed scanner, Anal Biochem, 293 (2001) 224-31),
chemiluminescene such as described by Huang, R. P. et al. (Huang, R. P., Detection of multiple proteins in an antibody-based protein microarray system, J Immunol Methods, 255 (2001) 1-13), Cheek, B. J., et. al. (Cheek, B. J., Steel, A. B., Torres, M. P., Yu, Y.-L., and Yang, H., Chemiluminescence Detection for Hybridization Assays on Flow-thru Chip, a Three-Dimensional Microchannel Biochip, Analytical Chemistry, 73 (2001) 5777-5783), Eggers, M. et. al. (Eggers, M., Hogan, M., Reich, R. K., Lamture, J., Ehrlich, D., Hollis, M., Kosicki, B., Powdrill, T., Beattie, K., Smith, S., and et al., A microchip for quantitative detection of molecules utilizing luminescent and radioisotope reporter groups, Biotechniques, 17 (1994) 516-25).

Mutation detection using these methods would all benefit by gradient stringency as described above prior to detection and analysis.

The dynamic range of detection methods based on CCD cameras, computer scanner and digital cameras to visualise chromagens are about 10-20-fold while the dynamic range of chemiluminesence is about 100 to 1000-fold depending on detection systems. This is much worse than for instance fluorescence with a dynamic range of about 100000-fold. Genotyping using a allel specific hybridisation array such as that shown in FIG. 16 shows that for fluorescence, one condition can (even if not optimal), be applied for detection of all sites. Detection using absorbance at one condition will however result in misclassification (FIG. 22a) of the genotype. There are three possibilities that can happen when detecting spots with different amount of bound probes using a detection systems with limited dynamic range (see FIG. 22a). (1) the wild type spot and the corresponding mutant spot does not give any signal and thus will give a null results, (2) it is over exposed resulting in the wildtype probe and the corresponding mutant probe giving similar and very strong signals and finally (3) wild type and mutants probes that function at the actual stringency. In contrast detection systems with large dynamic range and better sensitivity will without trouble genotype the hypothetical patient as homozygote wildtype at all positions. However, the same genotyping results as fluorescent detection can be obtained using colorimetric or other detection systems with low dynamic range if multiple stringencies are employed (FIG. 22b). It is apparent from FIG. 22b that at some stringency, the probes are working and the readout will be a homozygote wildtype. A signature of heterozygotes is that the ratio of the signal between wild type and the respective mutant probe is equal irrespective of stringency applied while homozygotes can at low stringencies applied show equal signal on both the wild type and the corresponding mutant probe. However, the ratio of the signal between wildtype probe and the corresponding mutant probe will always change at higher stringency if the patient is homozygote.

### Example 3

### Effect on Assay Robustness Using Non Melting Temperature Matched Probes and a Gradient If Condition Over and Array.

The effects of the multi thermal chip was tested using a set of melting temperature (Tm) matched probes and a set of non- melting temperature matched shorter probes for mutation detection in the beta-globin gene. The Tm matched set of probes was designed using thermodynamic models. The probes included in this set varied in length between 15 nt and 21 n and had a theoretical Tm of 50° C. (Described in (Petersen, J., Stangegaard, M., Birgens, H. & Dufva, M. Detection of mutations in the beta-globin gene by colorimetric staining of DNA microarrays visualized by a flatbed scanner. Anal Biochem (2006)). A short probes set with non Tm matched probes was also constructed, where the only requirements was that the probes were as short as possible but still gave signals in hybridization reactions. These probes were mostly 12-17 nt long and had melting temperatures in the range 35-57° C. (the probe set is described in (Dufva, M., Petersen, J., Stoltenborg, M., Birgens, H. & Christensen, C. B. Detection of mutations using microarrays of poly(C)10-poly(T)10 modified DNA probes immobilized on agarose films. Anal Biochem 352, 188-97 (2006))). Both probe sets where printed in eight identical subarrays on agarose film coated slides (Dufva, M., Petronis, S., Jensen, L. B., Krag, C. & Christensen, C. B. Characterization of an inexpensive, nontoxic, and highly sensitive microarray substrate. Biotechniques 37, 286-92, 294, 296 (2004)). The slides were hybridized with amplified DNA samples carrying different beta-thalassemia mutation as well as, controls with no mutations. The hybridized slides were mounted in the device and the subarrays were washed for 30 minutes. The optimal single temperature of the Tm set was determined by t-tests between homozygote wild types and heterozygotes for each mutation investigated and showed that the most robust genotype was obtained when washing at 37° C. The Tm matched set could be used for genotyping and no misclassification was observed for the totally of 279 genotyping (FIG. 23A). However, heterozygotes spread over a large range (ratio 0.33 to 0.73). It was apparent that even though the Tm matched set was designed to work at one and one only condition, the probes pair had different optimum operation temperature ranging from 25-40° C. (FIG. 23B). Applying optimal temperature for each probe pair led to slightly lower variance of the heterozygotes. For example the misclassification rate of the CD27/CD28 was 1 in 10¹² at 37° C. and 1 in 10¹⁸ at 40° C. Two probe pair for analysis of CD24 and CD27/CD28 resulted in high ratios for heterozygotes at all conditions. The probes for CD24 were 19-21 nt long which may explain the low discrimination power of this probe set. 13 nt probes could however be used to analyse CD24 mutation (FIG. 23 C). Similarly, CD27/28 mutation could be accurately analysed using shorter probes than those employed in the Tm matched set. The shorter probes did however function at 22° C. and 37° C. respectively (FIG. 3C). Taken together the variation of heterozygotes could almost be halved if fluidics device creating a temperature gradient over a microarray was used in combination with probes with different working optimum. A multi-condition assay can thus be used to enable simple calling of genotypes where homozygote mutated ranges between 0.7 and 1, heterozygotes between 0.35 and 0.65 and homozygote mutated less than 0.3 (FIG. 3C). Such constriction of the respective genotype is necessary for accurate genotyping of homozygote mutate, heterozygotes and homozygote wild type.

### Example 4

### Using the Device as a Combined Hybridization and Washing Station.

The device can also be used for combining hybridization and subsequent multi stringency washing. There are several possibilities to combine hybridization with stringency wash. One option is simply to place the hybridization solution on the wells of the base and subsequently mount the DNA microarray support on the device. The DNA microarray is then exposed to the hybridization solution containing target. The hybridization temperature can be set for all chambers to be equal so that each chamber provides the same hybridization condition. After a given time, the gradient of temperature or chemicals can be created by starting the pumping systems. The target is flushed out and the microarray support is subsequently washed at different stringencies. The other possibility for combining hybridization and multistringency washing utilized the fluidics system to load the chambers with hybridization chambers after the DNA microarray support has been mounted. A pump can also be programmed to provide rocking motions (altering between pushing and sucking) that together with special microstructures (such as herring bone mixers) in the bottom of the chambers to create mixing during hybridization. The mixing will speed up hybridization, give higher signal and give more even hybridization over the arrays. The hybridization can be performed at a particular temperature for all chambers. After hybridization, the microarray solid support is washed at different condition either using a chemical or a thermal gradient. The latter way of performing hybridization is particularly suitable for combination with the thermal gradient because the fluidics is simple.

1. A device for performing an assay, comprising means for receiving one or more replaceable solid support (40) onto which chemical entities (41) are attached, said device comprising a base, one or more inlet, one or more outlet; said base, and said solid support defining, when operatively connected, one or more chambers each having an area of minimum 3×3 mm² comprising said chemical entities, said inlet and outlet and chambers being in fluid connection; means for providing differing chemical conditions in each chamber; and means for providing a defined temperature either in each chamber or in one or more groups of chambers, where each group is related to one solid support.

2. The device according to claim 1, wherein the means for providing a defined temperature comprises an environmental chamber in which the whole device is enclosed providing constant temperature for all chambers.

3. The device according to claim 1, wherein the device comprises one or more temperature-regulator for providing a spatial temperature gradient between the chambers.

4. The device according to claim 3, wherein the one or more temperature-regulator is/are made of one or more electrically conductive materials selected from the group consisting of resistive metals, conductive ceramic, conductive polymer, electrolytes and semiconductor.

5. The device according to claim 4, wherein the temperature-regulator is a heater made of a metal or alloy component.

6. The device according to claim 3, wherein the temperature regulator is in the form of a wire, coil, patterned planar structure, thin film or electronic device.

7. The device according to claim 3, wherein the temperature regulators are located in the base and correspond to the areas forming the chambers.

8. The device according to claim 1, wherein the means for providing differing chemical conditions in each chamber provides a gradient of chemical concentration.

9. The device according to claim 8, wherein the means for providing differing chemical conditions in each chamber provides a linear or a non-linear concentration gradient.

10. The device according to claim 1, wherein the means for providing differing chemical conditions in each chamber (21) comprises a micro channel network for providing a chemical concentration gradient in a fluid.

11. The device according to claim , wherein the means for providing differing chemical conditions in each chamber comprises a series of entering channels for leading predefined chemical solutions of fluid into each chamber of the device.

12. The device according to claim 10, wherein the fluid is transported to the chambers by means of gravity or by pumps.

13. The device according to claim 10, wherein the fluid is either pumped or drawn through the chambers.

14. The device according to claim 1, wherein the assay is a bioassay.

15. The device according to claim 14, wherein the bioassay is based on one or a combination of chemical compounds selected from the group consisting of proteins, peptides, oligonucleotides, carbohydrates, polysaccharides, lipids, antibodies, antigens, cells and cell parts.

16. The device according to claim 1, wherein the assay is a chemical assay.

17. The device according to claim 16, wherein the chemical assay is based on one or more compounds comprising pesticides, pollution factors, toxic compounds, explosives, drug or pharmaceutical compositions.

18. The device according to claim 1, further comprising clamping means for pressing the base against the solid support, where the base comprises several layers where one layer provides feed lines, another layer provides a fluid connection, yet another layer provides a gasket, yet another layer defines the basis for the solid support and one layer provides a lid or cover.

19. The device according to claim 1, further comprising a sealer for providing a hermetic seal between the base and the solid support.

20. The device according to claim 17, further comprising a top part, for applying even pressure on the solid support when inserted in the device.

21. The device according to claim 1, wherein said base, is made of a polymer material, ceramic material or a combination thereof.

22. The device according to claim 21, wherein the polymer material is any one of polymethyl-methacrylate, cyclo-olefin copolymer, polystyrene, polycarbonate, polyvinyl chloride, acrylics, polytetrafluoro ethylene, epoxy, polypropylene, polysulfone, polyethylene, nylon based polymers and co-polymers or composites.

23. The device according to claim 21, wherein the ceramic material is any one of glass, silicon, porcelain or quartz or any combination thereof.

24. The device according to claim 1, wherein the solid support is made from glass, polymers, silicon, metals, ceramics and has the format of a microscope slide, a chip or a microtiter plate.

25. The device according to claim 24, wherein the solid support is a slide comprising an array of chemical entities on its surface.

26. The device according to claim 25, wherein the array comprises a repeated pattern of chemical entities, such that when said solid support is inserted in the device each chamber comprises the same number and composition of chemical entities.

27. The device according to claim 1, wherein a solid support relates to 3-30 chambers where each chamber is characterised by one or more parameters.

28. The device according to claim 1, which is reusable.

29. The device according to claim 1, which provides different fluid phase conditions.

30. The device according to claim 1, wherein the chambers each have a volume >20 μl.

31. The device according to claim 1, wherein the chambers each have a height >50 μm.

32. The device according to claim 1, wherein the chambers are provided with individual or common shaker units for loosening or transporting formed bubbles.

33. The device according to claim 1, wherein a bubble trap is provided in front of each chamber.

34. The device according to claim 1, wherein one or more of the inner surfaces of the chambers are provided with protruding or intruding topographic structures on the surface in order to distribute the flow or create turbulence when fluid is flowing in the chamber.

35. The device according to claim 34, wherein the structures are formed in the bottom of the chamber as one or more rhombs or as one or more successive arrow heads.

36. The device according to claim 1, wherein each chamber is fully or partly filled with a defined amount of fluid where after the defined amount of fluid is pulsated across the chemical entities of the solid support.

37. The device according to claim 1, wherein the chemical entities positioned on the solid support can get into direct contact with the fluid inside the chamber.

38. A method of conducting an assay comprising the steps of:
providing a device according to claim 1,
inserting a solid support onto which chemical entities have been attached in areas corresponding to chambers in said device,
transferring a fluid to and/or through the chambers of the device
optionally providing exactly defined and varying chemical conditions in each chamber in the device,
optionally setting exactly defined temperature in each chamber of the device,
comparing the chemical entities or bound ligands in one chamber to the chemical entities or bound ligands in another chamber, said chemical entities having been in contact with the fluid.

39. The method according to claim 38, wherein the fluid transferred to and/or through the chambers of the device is a fluid for removing analyte by stringent washing.

40. The method according to claim 38, wherein a first fluid transferred to and/or through the chambers of the device is a fluid containing an analyte for hybridisation and after clearing the chambers for the first fluid a second fluid for removing bound analyte by stringent washing is transferred to and/or through the chambers.

41. The method according to claim 38, wherein the chemical entities are identified by a colorimetric method.

42-43. (canceled)

44. A kit comprising a device according to claim 1.

45. The device according to claim 5, wherein the heater is made of iron, copper, platinum, aluminium, nickel, gold, wolfram, tungsten, or chromium metals or alloys.

46. The device according to claim 9, wherein each chamber provides a logarithmic concentration gradient.

47. The device according to claim 18, wherein the one layer that provides feed lines also provides a mixing tree and/or temperature controllers.
